(19) United States
(12) Patent Application Publication
(10) Pub. No.: US 2025/0101442 A1
(43) Pub. Date: Mar. 27, 2025

(54) COMPOSITIONS AND METHODS FOR ENHANCED PROTEIN PRODUCTION IN GRAM-POSITIVE BACTERIAL CELLS

(71) Applicant: DANISCO US INC., Palo Alto, CA (US)

(72) Inventors: Christopher Webb, Menlo Park, CA (US); Cristina Bongiorni, Fremont, CA (US); Shannon Del Chase, Mountain View, CA (US)

(21) Appl. No.: 18/728,353

(22) PCT Filed: Jan. 10, 2023
(86) PCT No.: PCT/US2023/060360

§ 371 Date: Jul. 11, 2024

Related U.S. Application Data
(60) Provisional application No. 63/299,159, filed on Jan. 13, 2022.

(51) Int. Cl. C12N 15/75; C07K 14/32; C12P 21/02

(52) U.S. Cl. CPC C12N 15/75; C07K 14/32; C12P 21/02

(57) ABSTRACT

The present disclosure is generally related to Gram-positive bacterial strains comprising enhanced protein productivity phenotypes. Certain aspects are therefore related to compositions and methods for constructing recombinant (modified) Gram-positive bacterial strains for the enhanced production of proteins of interest.

### CROSS REFERENCE TO RELATED APPLICATIONS

This application claims benefit to U.S. Provisional Patent Application No. 63/299,159, filed Jan. 13, 2022, which is incorporated herein by referenced in its entirety.

### FIELD

The present disclosure is generally related to the fields of bacteriology, microbiology, genetics, molecular biology, enzymology, industrial protein production the like. Certain embodiments of the disclosure are related to Gram-positive bacterial cells comprising enhanced protein productivity phenotypes, compositions and methods for constructing recombinant Gram-positive bacterial cells, and the like.

### REFERENCE TO A SEQUENCE LISTING

The contents of the electronic submission of the text file Sequence Listing, named “NB41845-WO-PCT_SequenceListing.xml” was created on Jan. 6, 2023 and is 35 KB in size, which is hereby incorporated by reference in its entirety.

### BACKGROUND

Gram-positive bacteria such as Bacillus subtilis, Bacillus licheniformis, Bacillus amyloliquefaciens and the like are frequently used as microbial factories for the production of industrial relevant proteins, due to their excellent fermentation properties and high yields (e.g., up to 25 grams per liter culture; Van Dijl and Hecker, 2013). For example, Bacillus sp. host cells are well known for their production of enzymes (e.g., amylases, cellulases, mannanases, pectate lysases, proteases, pullulanases, etc.) necessary for food, textile, laundry, medical instrument cleaning, pharmaceutical industries and the like. Because these non-pathogenic Gram-positive bacteria produce proteins that completely lack toxic by-products (e.g., lipopolysaccharides; LPS, also known as endotoxins) they have obtained the “Qualified Presumption of Safety” (QPS) status of the European Food Safety Authority (EFSA), and many of their products gained a “Generally Recognized As Safe” (GRAS) status from the US Food and Drug Administration (Olempska-Beer et al., 2006; Earl et al., 2008; Caspers et al., 2010).

Thus, the production of proteins (e.g., enzymes, antibodies, receptors, etc.) via microbial host cells is of particular interest in the biotechnological arts. Likewise, the optimization of Bacillus host cells for the production and secretion of one or more protein(s) of interest is of high relevance, particularly in the industrial biotechnology setting, wherein small improvements in protein yield are quite significant when the protein is produced in large industrial quantities. For example, the expression of many heterologous proteins can still be challenging and unpredictable with respect to yield and the like. As described hereinafter, the present disclosure is related to the highly desirable and unmet needs for obtaining and constructing Gram-positive cells (e.g., protein production hosts) having enhanced protein production capabilities.

### SUMMARY

As generally described herein, Applicant has surprisingly observed that overexpression of a yvyD gene is particularly relevant to the enhanced production of proteins of interest in Gram-positive bacterial cells. Thus, certain aspects of the disclosure are related to compositions and methods for producing proteins of interest. More particularly, certain embodiments of the disclosure provide, inter alia, recombinant (modified) Gram-positive bacterial cells (strains) overexpressing a yvyD gene, recombinant Gram-positive bacterial cells expressing/producing one or more proteins of interest, recombinant Gram-positive bacterial cells expressing proteins of interest and overexpressing a yvyD gene, polynucleotide constructs (e.g., plasmids, vectors, expression cassettes, etc.) suitable for introducing into bacterial strains and the like. Certain aspects are therefore related to recombinant Gram-positive bacterial cells overexpressing a yvyD gene, wherein the recombinant cells produce increased amounts of proteins of interest, demonstrating enhanced specific productivities (Qp) of proteins produced when cultivated under suitable conditions, demonstrating enhanced carbon efficiency of proteins produced when cultivated under suitable conditions, and the like.

### BRIEF DESCRIPTION OF DRAWINGS

FIG. 1 shows schematic maps of integration cassettes, wherein FIG. 1A shows integration cassette “yvzG-yvyD intergene::lox-SpecR-lox-PspoVG-yvyD” (SEQ ID NO: 19) and FIG. 1B shows integration cassette “yvzG-yvyD intergene::lox-SpecR-lox-Phbs-yvyD” (SEQ ID NO: 20). For example, as shown in FIG. 1A, integration cassette “yvzG-yvyD intergene::lox-SpecR-lox-PspoVG-yvyD” (SEQ ID NO: 19) comprises the upstream (5′) “fliS” sequence followed by “fliT” sequence followed by “yvzG” gene sequence, which are upstream of the lox-SpecR-lox sequence followed by the “PspoVG-yvyD” sequence followed by the downstream (3′) “secA” sequence. As shown, the FIG. 1B, integration cassette “yvzG-yvyD intergene::lox-SpecR-lox-Phbs-yvyD” (SEQ ID NO: 20) comprises the upstream (5′) “fliS” sequence followed by “fliT” sequence followed by “yvzG” gene sequence, which are upstream of the lox-SpecR-lox sequence followed by the “Phbs-spoVGSD-yvyD” sequence followed by the downstream (3′) “secA” sequence.

FIG. 2 shows the protease activity (arbitrary units) of sample aliquots taken at the specified time points (Example 3). The protease productivity (Protease-1) of control strain (2× Protease-1) and the modified strain (2× Protease-1+PspoVG-yvyD) fermented under the same conditions were compared (FIG. 2).

FIG. 3 shows the protease activity (arbitrary units) of sample aliquots taken at the specified time points (Example 3). The protease productivity (Protease-2) of control strain (2× Protease-2) and the modified strain (2× Protease-2+PspoVG-yvyD) fermented under the same conditions were compared (FIG. 3).

FIG. 4 shows the protease activity (arbitrary units) of sample aliquots taken at the specified time points (Example 3). The protease productivity (Protease-2) of control strain (2× Protease-2) and the modified strain (2× Protease-2+Phbs-yvyD) fermented under the same conditions were compared (FIG. 4).

FIG. 5 shows the protease activity (arbitrary units) of sample aliquots taken at the specified time points (Example 3). The protease productivity (Protease-3) of control strain (2× Protease-3) and the modified strain (2× Protease-3+Phbs-yvyD) fermented under the same conditions were compared (FIG. 5).

FIG. 6 presents the amino acid sequence of the native B. subtilis YvyD protein (SEQ ID NO: 26). As presented in FIG. 6, the YvyD protein (SEQ ID NO: 26) comprises an N-terminal conserved RaiA superfamily domain (SEQ ID NO: 27) shown with underlined amino acid residues; and a C-terminal conserved Ribosome S30AE_C superfamily domain (SEQ ID NO: 28) indicated with bold amino acid residues.

FIG. 7 shows the nucleic acid sequence of the Phbs promoter region set forth in SEQ ID NO: 29. In particular, the Phbs promoter region (SEQ ID NO: 29; FIG. 1A) comprises an upstream (5′) hbs promoter sequence (SEQ ID NO: 22; FIG. 7B) operably linked to a downstream (3′) spoVG Shine-Dalgarno (SD) sequence (SEQ ID NO: 25; FIG. 7C). As presented in FIG. 1A, the nucleotides of the hbs promoter sequence (Phbs; SEQ ID NO: 22) are underlined and the nucleotides of the Shine-Dalgarno sequence (SD; SEQ ID NO: 25) are bold.

### BRIEF DESCRIPTION OF THE BIOLOGICAL SEQUENCES

SEQ ID NO: 1 is a synthetic DNA sequence of a primer named “343”.

SEQ ID NO: 2 is a synthetic DNA sequence of a primer named “402”.

SEQ ID NO: 3 is a synthetic DNA sequence of a primer named “400”.

SEQ ID NO: 4 is a synthetic DNA sequence of a primer named “370”.

SEQ ID NO: 5 is a synthetic DNA sequence of a primer named “539”.

SEQ ID NO: 6 is a synthetic DNA sequence of a primer named “246”.

SEQ ID NO: 7 is a synthetic DNA sequence of a primer named “540”.

SEQ ID NO: 8 is a synthetic DNA sequence of a primer named “754”.

SEQ ID NO: 9 is a synthetic DNA sequence (36 bp) primer of the spoVG promoter region.

SEQ ID NO: 10 is a synthetic DNA sequence of a primer named “675”.

SEQ ID NO: 11 is a synthetic DNA sequence of a primer named “307”.

SEQ ID NO: 12 is a synthetic DNA sequence of a primer named “674”.

SEQ ID NO: 13 is a synthetic DNA sequence of a primer named “345”.

SEQ ID NO: 14 is a synthetic DNA sequence of a primer named “348”.

SEQ ID NO: 15 is a synthetic DNA sequence of a primer named “346”.

SEQ ID NO: 16 is a synthetic DNA sequence of a primer named “300”.

SEQ ID NO: 17 is a synthetic DNA sequence of a primer named “573”.

SEQ ID NO: 18 is the open reading frame (DNA) sequence of the yvyD gene CDS.

SEQ ID NO: 19 is a polynucleotide (integration) cassette named “yvzG-yvyD intergene::lox-SpecR-lox-PspoVG-yvyD”.

SEQ ID NO: 20 is a polynucleotide (integration) cassette named “yvzG-yvyD intergene::lox-SpecR-lox-Phbs-yvyD”.

SEQ ID NO: 21 is a spoVG promoter region (PspoVG) comprising the upstream (5′) spoVG promoter operably linked to the downstream (3′) spoVG Shine-Dalgarno (SD) sequence.

SEQ ID NO: 22 is a hbs promoter (Phbs) sequence.

SEQ ID NO: 23 is a B. subtilis yvyD gene (DNA) sequence comprising the yvyD promoter region and the yvyD gene coding sequence (CDS; i.e., SEQ ID NO: 18).

SEQ ID NO: 24 is the DNA sequence of the upstream (5′) yvyD promoter region of the yvyD gene set forth in SEQ ID NO: 23.

SEQ ID NO: 25 is a DNA sequence comprising a native spoVG Shine-Dalgarno (SD) sequence.

SEQ ID NO: 26 is the amino acid sequence of the B. subtilis native YvyD protein.

SEQ ID NO: 27 is the amino acid sequence of the N-terminal conserved RaiA superfamily domain of the native YvyD protein (SEQ ID NO: 26).

SEQ ID NO: 28 is the amino acid sequence of the C-terminal conserved Ribosome S30AE_C superfamily domain of the native YvyD protein (SEQ ID NO: 26).

SEQ ID NO: 29 is a hbs promoter region sequence comprising the hbs promoter (Phbs) sequence (SEQ ID NO: 22) operably linked to the SD sequence (SEQ ID NO: 25).

### DETAILED DESCRIPTION

As described herein, certain embodiments of the disclosure are related to compositions and methods for enhanced protein production in Gram-positive bacterial (host) cells/strains. More particularly, as set forth hereinafter, and further described in the Examples below, the recombinant (genetically modified) Gram-positive bacterial cells of the disclosure are particularly useful for the enhanced production of proteins of interest. Certain embodiments of the disclosure are related to, inter alia, recombinant polynucleotides increasing yvyD gene expression in Gram-positive bacterial cells, recombinant Gram-positive cells overexpressing a yvyD gene coding sequence (CDS; e.g., yvyD ORF; SEQ ID NO: 18), recombinant Gram-positive cells overexpressing a yvyD gene CDS and expressing one or more (multiple) copies of genes encoding proteins of interest, and the like.

### I. Definitions

In view of the recombinant (modified) cells of the disclosure and methods thereof described herein, the following terms and phrases are defined. Terms not defined herein should be accorded their ordinary meaning as used in the art.

Unless defined otherwise, all technical and scientific terms used herein have the same meaning as commonly understood by one of ordinary skill in the art to which the present compositions and methods apply. Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof. For example, in certain aspects, a Gram-positive bacterial “control cell/strain” produces a protein of interest, but “does not” comprise an overexpressed yvyD gene.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the phrases “Gram-positive bacteria”, Gram-positive cells” “Gram-positive bacterial strains”, and/or “Gram positive bacterial cells” have the same meaning as used in the art. For example, Gram-positive bacterial cells include all strains of Actinobacteria and Firmicutes. In certain embodiments, such Gram-positive bacteria are of the classes Bacilli, Clostridia and Mollicutes. As used herein, the terms “recombinant” or “non-natural” refer to an organism, microorganism, cell, nucleic acid molecule, or vector that has at least one engineered genetic alteration, or has been modified by the introduction of a heterologous nucleic acid molecule, or refer to a cell (e.g., a microbial cell) that has been altered such that the expression of a heterologous or endogenous nucleic acid molecule or gene can be controlled. Recombinant also refers to a cell that is derived from a non-natural cell or is progeny of a non-natural cell having one or more such modifications. Genetic alterations include, for example, modifications introducing expressible nucleic acid molecules encoding proteins, or other nucleic acid molecule additions, deletions, substitutions or other functional alteration of a cell's genetic material. For example, recombinant cells may express genes or other nucleic acid molecules that are not found in identical or homologous form within a native (wild-type) cell (e.g., a fusion or chimeric protein), or may provide an altered expression pattern of endogenous genes, such as being over-expressed, under-expressed, minimally expressed, or not expressed at all. “Recombination”. “recombining” or generating a “recombined” nucleic acid is generally the assembly of two or more nucleic acid fragments wherein the assembly gives rise to a chimeric gene.

As used herein, the terms “yvyD” or “wvyD gene” refer to a gene (or a yvyD gene homologue) encoding a “YvyD” protein (or YvyD protein homologue). As generally set forth below in Section II, the YvyD protein is believed to function as a “general stress factor” or “ribosome hibernation promotion factor”. The term yvyD gene includes synonymous names such as the “hpf” gene and/or the “yvil” gene.

As used herein, the phrases “overexpression of yvyD” or “increased yvyD expression” means increased expression of a yvyD gene coding sequence (CDS). In certain aspects, a yvyD gene CDS comprises at least 80% sequence identity to the yvyD open reading frame (ORF; SEQ ID NO: 18). For example, in certain aspects, increased expression of yvyD may be performed by substituting (replacing) the native upstream (5′) “yvyD promoter region” with a suitable heterologous (replacement) promoter region, wherein expression of the downstream yvyD gene CDS is controlled (increased) by the heterologous (replacement) promoter. In certain aspects, a yvyD gene CDS comprises at least about 50% to 100% identity to the wvyD ORF of SEQ ID NO: 18. In certain embodiments, a yvyD gene CDS comprises at least 80%, 81%, 82%, 83%, 84%, 85%, 86%, 87%, 88%, 89%, 90%, 91%, 92%, 93%, 94%, 95%, 96%, 97%, 98%, 99% or 100% identity to the wyD ORF of SEQ ID NO: 18.

As used herein, the phrase native “yvyD promoter region” (abbreviated, “PyvyD”) means a “yvyD gene promoter” comprising at least about 60% to 100% sequence identity to the native B. subtilis yvyD gene promoter region sequence of SEQ ID NO: 24. In certain embodiments, a yvyD gene promoter region (PyvyD) comprises at least 85%, 86%, 87%, 88%, 89%, 90%, 91%, 92%, 93%, 94%, 95%, 96%, 97%, 98%, 99% or 100% identity to the PyvyD of SEQ ID NO: 24.

As used herein, the term “hbs promoter sequence” (abbreviated, “Phbs”) refers to a nucleic acid comprising at least about 60% to 100% identity to SEQ ID NO: 22. In certain embodiments, a hbs promoter (Phbs) sequence comprises at least 85%, 86%, 87%, 88%, 89%, 90%, 91%, 92%, 93%, 94%, 95%, 96%, 97%, 98%, 99% or 100% identity to SEQ ID NO: 22.

As used herein, the term “spoVG Shine-Dalgarno (SD) sequence” refers to a nucleic acid comprising at least about 60% to 100% identity to SEQ ID NO: 25. In certain aspects, a spoVG SD sequence comprises at least 85%, 86%, 87%, 88%, 89%, 90%, 91%, 92%, 93%, 94%, 95%, 96%, 97%, 98%, 99% or 100% identity to SEQ ID NO: 25.

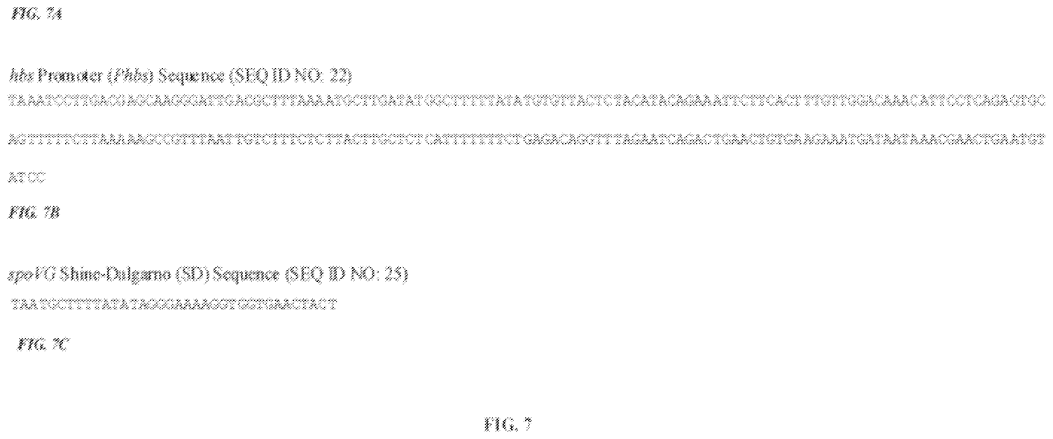

As used herein, the term “hbs promoter region sequence” (abbreviated, “Phbs region sequence”) refers to a nucleic acid comprising at least about 60% to 100% identity to SEQ ID NO: 29. For example, as shown in FIG. 7A, a Phbs promoter region (SEQ ID NO: 29) sequence comprises the Phbs promoter (SEQ ID NO: 22) positioned upstream and in operable combination with the SD sequence (SEQ ID NO: 25). Thus, in certain embodiments, a Phbs promoter region comprises at least 85%, 86%, 87%, 88%, 89%, 90%, 91%, 92%, 93%, 94%, 95%, 96%, 97%, 98%, 99% or 100% identity to SEQ ID NO: 29.

As used herein, the term “spoVG promoter region” (abbreviated, “PspoVG”) refers to a nucleic acid comprising at least about 60-100% identity to SEQ ID NO: 21. In certain embodiments, a spoVG promoter region (PspoVG) comprises at least 85%, 86%, 87%, 88%, 89%, 90%, 91%, 92%, 93%, 94%, 95%, 96%, 97%, 98%, 99% or 100% identity to the PspoVG of SEQ ID NO: 21.

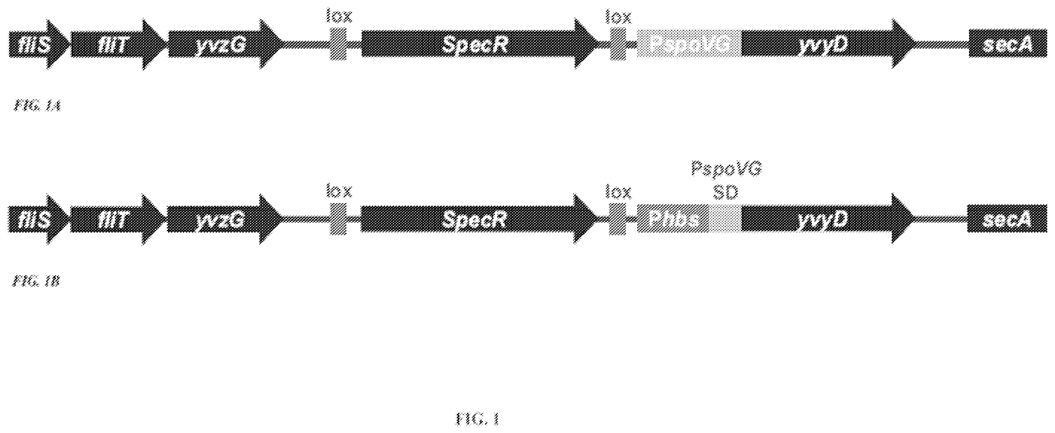

As used herein, exemplary “yvyD gene overexpression (integration) cassettes” are schematically presented in FIG. 1, wherein integration cassettes named “yvzG yvyD intergene::lox-SpecR-lox-PspoVG-yvyD” (FIG. 1A; SEQ ID NO: 19) and “yvzG yvyD intergene::lox-SpecR-lox-Phbs-yvyD” (FIG. 1B; SEQ ID NO: 20) are shown.

As used herein, the term “promoter swap”, when used in phrases such as “increased expression of a yvyD gene by a ‘promoter swap’ integration”, means replacing (substituting) the yvyD gene promoter region sequence upstream (5′) of the native yvyD gene coding sequence (CDS) with a “heterologous (replacement) promoter” sequence, wherein the promoter swapped yvyD gene CDS is expressed (overexpressed) under the control of the heterologous (replacement) promoter.

As used herein, genes encoding exemplary reporter proteases may be recited as “protease-1” gene, “protease-2” gene, “protease-3” gene, and the like, wherein the encoded proteases are recited as “Protease-1”, “Protease-2”, “Protease-3” and the like, respectively.

As used herein, phrases such as, “two (2) copies protease-1” and “encoding two (2) copies of Protease-1” may be abbreviated as “2× protease-1” and “2× Protease-1”, respectively, “two (2) copies protease-2” and “encoding two (2) copies of Protease-2” may be abbreviated as “2× protease-2” and “2× Protease-2”, respectively”, “two (2) copies protease-3” and “encoding two (2) copies of Protease-3” may be abbreviated as “2× protease-1” and “2× Protease-1”, respectively, and the like.

As used herein, a reporter protease named “Protease-1” refers to a variant Bacillus lentus subtilisin (protease) described in PCT Publication No. WO2012/151534 (incorporated herein by reference in its entirety).

As used herein, a reporter protease named “Protease-2” refers to a variant Bacillus gibsonii protease described in PCT Publication No. WO2020/243738 (incorporated herein by reference in its entirety).

As used herein, a reporter protease named “Protease-3” refers to a variant Bacillus amyloliquefaciens BPN′ protease described in PCT Publication No. WO2011/72099A (incorporated herein by reference in its entirety).

Thus, in certain aspects, Gran-positive cells of the disclosure comprise an endogenous yvyD gene encoding a native YvyD protein, wherein the yvyD gene comprises about 50% to 100% identity to the yvyD gene of SEQ ID NO: 23. In other embodiments, the yvyD gene comprises at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 23.

SEQ ID NO: 26 is the amino acid sequence of the B. subtilis native YvyD protein, SEQ ID NO: 27 is the amino acid sequence of the N-terminal conserved RaiA superfamily domain of the native YvyD protein (SEQ ID NO: 26) and SEQ ID NO: 28 is the amino acid sequence of the C-terminal conserved Ribosome S30AE_C superfamily domain of the native YvyD protein (SEQ ID NO: 26).

As used herein, a “host cell” refers to a cell that has the capacity to act as a host or expression vehicle for a newly introduced DNA sequence. Thus, in certain embodiments of the disclosure, the host cells are Gram-positive (e.g., Bacilli) and/or Gram-negative (e.g., E. coli) cells.

As used herein, the phrases a “modified Gram-positive cell” and/or a “Gram-positive (daughter) cell” refer to recombinant Gram-positive cells that comprises at least one genetic modification which is not present in the parent (control) Gram-positive cell from which the modified Gram-positive cell was derived. For example, when the expression/production of a protein of interest (POI) a Gram-positive “control” cell is being compared to the expression/production of the same POI in a “modified” (recombinant) cell, it will be understood that the “control” and “modified” cells are grown/cultivated/fermented under the same conditions (e.g., the same conditions such as media, temperature, pH and the like).

As used herein, “increasing” protein production or “increased” protein production is meant an increased amount of protein produced (e.g., a protein of interest). The protein may be produced inside the host cell, or secreted (or transported) into the culture medium. In certain embodiments, the protein of interest is produced (secreted) into the culture medium. Increased protein production may be detected for example, as higher maximal level of protein or enzymatic activity (e.g., such as protease activity, amylase activity, pullulanase activity, cellulase activity, and the like), or total extracellular protein produced as compared to the parental cell.

As used herein, the terms “modification” and “genetic modification” are used interchangeably and include: (a) the introduction, substitution, or removal of one or more nucleotides in a gene (or an ORF thereof), or the introduction, substitution, or removal of one or more nucleotides in a regulatory element required for the transcription or translation of the gene or ORF thereof. (b) a gene disruption, (c) a gene conversion, (d) a gene deletion, (e) the down-regulation of a gene, (f) specific mutagenesis and/or (g) random mutagenesis of any one or more the genes disclosed herein.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any steps involved in the production of the polypeptide including, but not limited to, transcription, post-transcriptional modification, translation, post-translational modification, secretion and the like.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein.

It is understood that the polynucleotides (or nucleic acid molecules) described herein include “genes”, “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (UTRs), and 3′-UTRs, as well as the coding sequence.

As used herein, the term “coding sequence” refers to a nucleotide sequence, which directly specifies the amino acid sequence of its (encoded) protein product. The boundaries of the coding sequence are generally determined by an open reading frame (hereinafter, “ORF”), which usually begins with an ATG start codon. The coding sequence typically includes DNA, cDNA, and recombinant nucleotide sequences.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “a functional promoter sequence controlling the expression of a gene of interest (or open reading frame thereof) linked to the gene of interest's protein coding sequence” refers to a promoter sequence which controls the transcription and translation of the coding sequence in Bacillus. For example, in certain embodiments, the present disclosure is directed to a polynucleotide comprising a 5′ promoter (or 5′ promoter region, or tandem 5′ promoters and the like), wherein the promoter region is operably linked to a nucleic acid sequence (e.g., an ORF) encoding a protein.

As used herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As used herein, the term “introducing”, as used in phrases such as introducing into a Gram-positive bacterial cell a gene, a polynucleotide, a vector, a cassette, and the like, includes methods known in the art for introducing polynucleotides into a cell, including, but not limited to protoplast fusion, natural or artificial transformation (e.g., calcium chloride, electroporation), transduction, transfection, conjugation and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). Transformation therefore generally refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein, “disruption of a gene” or a “gene disruption”, are used interchangeably and refer broadly to any genetic modification that substantially prevents a host cell from producing a functional gene product (e.g., a protein). Thus, as used herein, a gene disruption includes, but is not limited to, frameshift mutations, premature stop codons (i.e., such that a functional protein is not made), substitutions eliminating or reducing activity of the protein internal deletions (such that a functional protein is not made), insertions disrupting the coding sequence, mutations removing the operable link between a native promoter required for transcription and the open reading frame, and the like.

As used herein “an incoming sequence” refers to a DNA sequence that is introduced into the Gram-positive bacterial cell chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a nonfunctional gene or operon. In some embodiments, the non-functional sequence may be inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the Gram-positive bacterial cell chromosome. More specifically, a homology box is an upstream or downstream region having between about 80 and 100% sequence identity, between about 90 and 100% sequence identity, or between about 95 and 100% sequence identity with the immediate flanking coding region of a gene, or part of a gene to be deleted, disrupted, inactivated, down-regulated and the like, according to the invention. These sequences direct where in the Gram-positive bacterial cell chromosome a DNA construct is integrated and directs what part of the chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, the marker can be an antimicrobial resistance marker (e.g., amp^{R}, phleo^{R}, speck, kan^{R}, ery^{R}, tet^{R}, cmp^{R} and neo^{R}). In some embodiments, the present invention provides a chloramphenicol resistance gene (e.g., the gene present on pC194). This resistance gene is particularly useful in embodiments involving chromosomal amplification of chromosomally integrated cassettes and integrative plasmids. Other markers useful in accordance with the invention include, but are not limited to auxotrophic markers, such as serine, lysine, tryptophan; and detection markers, such as β-galactosidase.

As defined herein, a host cell “genome” and/or a Gram-positive bacterial cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell. in some embodiments plasmids exist in a parental cell and are lost in the daughter cell.

A used herein, a “transformation cassette” refers to a specific vector comprising a gene (or ORF thereof), and having elements in addition to the foreign gene that facilitate transformation of a particular host cell.

As used herein, the term “vector” refers to any nucleic acid that can be replicated (propagated) in cells and can carry new genes or DNA segments into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

An “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing mutations into the chromosome of a host cell through homologous recombination. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. For example, in certain embodiments, a parental Gram-positive (host) cell is modified (e.g., transformed) by introducing therein one or more “targeting vectors”.

As used herein, the term “protein of interest” or “POI” refers to a polypeptide of interest that is desired to be expressed in a Gram-positive bacterial cell. Thus, as used herein, a POI may be an enzyme, a substrate-binding protein, a surface-active protein, a structural protein, a receptor protein, a protein biologic, and the like. In certain embodiments, a modified Gram-positive cell of the disclosure produces an increased amount of a heterologous POI or an endogenous POI relative to its parent. In particular embodiments, an increased amount of a POI produced by a modified cell is at least a 0.5% increase, at least a 1.0% increase, at least a 5.0% increase, or a greater than 5.0% increase, relative to the parent.

Similarly, as defined herein, a “gene of interest” or “GOI” refers a nucleic acid sequence (e.g., a polynucleotide, gene, ORF) which encodes a POI. A “GOI” encoding a “POI” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., a acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer. As used herein, the term “foreign” gene(s) comprise native genes (or ORFs) inserted into a non-native organism and/or chimeric genes inserted into a native or non-native organism.

As defined herein, a “heterologous control sequence”, refers to a gene expression control sequence (e.g., a promoter or enhancer) which does not function in nature to regulate (control) the expression of the gene of interest. Generally, heterologous nucleic acid sequences are not endogenous (native) to the cell, or a part of the genome in which they are present, and have been added to the cell, by infection, transfection, transformation, microinjection, electroporation, and the like. A “heterologous” nucleic acid construct may contain a control sequence/DNA coding (ORF) sequence combination that is the same as, or different, from a control sequence/DNA coding sequence combination found in the native host cell.

As used herein, the terms “signal sequence” and “signal peptide” refer to a sequence of amino acid residues that may participate in the secretion or direct transport of a mature protein or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “derived” encompasses the terms “originated” “obtained,” “obtainable,” and “created,” and generally indicates that one specified material or composition finds its origin in another specified material or composition, or has features that can be described with reference to another specified material or composition.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using a computer program known in the art, such as “GAP” provided in the GCG program package (Program Manual for the Wisconsin Package, Version 8, August 1994, Genetics Computer Group, 575 Science Drive, Madison, Wisconsin, USA 53711) (Needleman and Wunsch, (1970). Using GAP with the following settings for DNA sequence comparison: GAP creation penalty of 5.0 and GAP extension penalty of 0.3.

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As defined herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, a “flanking sequence” refers to any sequence that is either upstream or downstream of the sequence being discussed (e.g., for genes A-B-C, gene B is flanked by the A and C gene sequences). In certain embodiments, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the Gram-positive bacterial cell chromosome. These sequences direct where in the chromosome the new construct gets integrated and what part of the chromosome will be replaced by the incoming sequence. In other embodiments, the 5′ and 3′ ends of a selective marker are flanked by a polynucleotide sequence comprising a section of the inactivating chromosomal segment. In some embodiments, a flanking sequence is present on only a single side (either 3′ or 5′), while in other embodiments, it is present on each side of the sequence being flanked.

### II. Overexpression of yvyD in Gram-Positive Bacterial Cells Enhances Protein Production

As briefly set forth above, the B. subtilis yvyD gene encodes a protein believed to function as a “general stress factor” or “ribosome hibernation promotion factor”. For example, as generally described in Drzewiecki et al. (1998), an analysis of 2-D protein gels for σ^{B}-dependent general stress proteins exhibiting atypical induction profiles identified a protein YvyD (previously named “Hst23”) as the product of the yvyD gene of B. subtilis. As described in this publication, in addition to the atypical σ^{B}-dependent, stress-inducible and starvation-inducible pattern, yvyD was also induced in response to amino acid depletion via σ^{H}-dependent promoter activation. In a study to elucidate the biological functions of small (p) ppGpp synthetases YjbM and YwaC of B. subtilis, Tagami et al. (2012) describe the construction of B. subtilis mutant strains (e.g., triple mutant; ΔrelA ΔyjbM AywaC), wherein YvyD protein is suggested to be essential for dimerization of the 70S ribosomes. More recently, cryo-EM structures of the B. subtilis 100S (Bs100S) particle revealed the binding site for the B. subtilis hibernation-promoting factor (BsHPF) named YvyD (Beckert et al., 2017). JP Patent Publication No. JP2009225711 has described deleting the B. subtilis yvyD gene, stating that the yvyD gene does not directly participate in the production of proteins of interest, and that YvyD protein is not necessary for the growth of microorganisms in ordinary industrial production media. Thus, in wild-type B. subtilis cells, YvyD is postulated to bind to inactive ribosomes during stress conditions to generally protect the ribosome.

As generally described herein and set forth in the Examples below, Applicant has surprisingly observed that overexpression of the yvyD gene is particularly relevant to the enhanced production of proteins of interest in Gram-positive bacterial cells. More particularly, as set forth in Example 1, Applicant designed and constructed yvyD (gene) overexpression (integration) cassettes (e.g., see FIG. 1) for introducing into exemplary Gram-positive (Bacillus) host cells. For example, the integration cassette fragments were designed to integrate at the yvzG-yvyD intergene region, replacing (substituting) the native yvyD gene promoter with a heterologous promoter (replacement). As shown in FIG. 1, the yvzG-yvyD integration cassettes comprise the promoter regions of spoVG (PspoVG; FIG. 1A, “PspoVG-yvyD”) or hbs (Phbs; FIG. 1B. “Phbs-yvyD”). In particular, as shown in FIG. 7A-7C, the Phbs promoter region sequence (SEQ ID NO: 29) comprises the upstream hbs promoter (Phbs) sequence (SEQ ID NO: 22) operably linked to the downstream spoVG SD sequence (SEQ ID NO: 25).

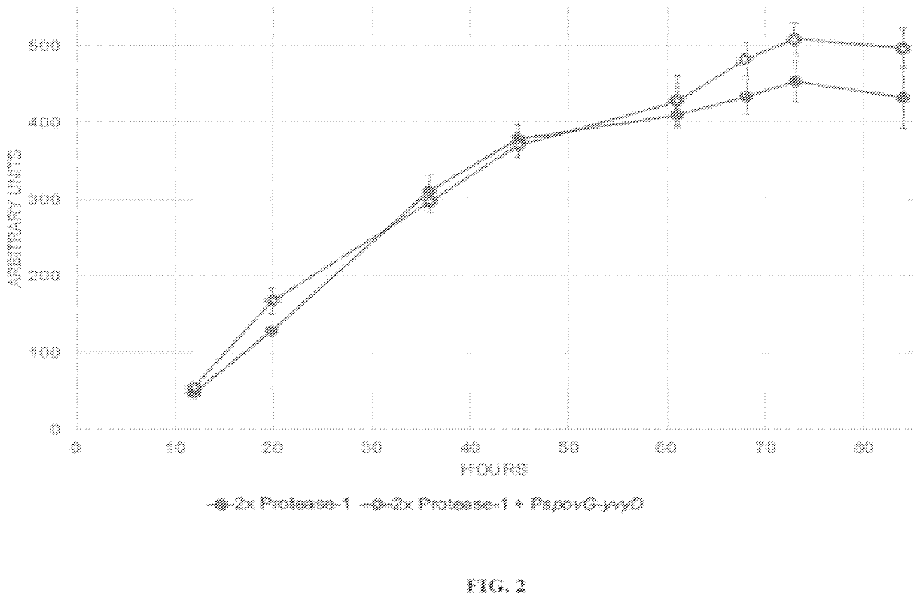

Example 2 further describes the construction of Gram-positive bacterial cells overexpressing yvyD. More specifically, the promoter swap integration cassettes described in Example 1 were introduced into recombinant Bacillus cells comprising two (2) copies of a gene encoding an exemplary reporter protein (e.g., 2 copies of gene encoding Protease-1, Protease-2, or Protease-3). As presented in Example 3. Applicant assessed the PspoVG-yvyD cassette for increased yvyD expression via reporter Protease-1 production in the Bacillus strains described in Example 2. More particularly, sample aliquots were taken from the 2× Protease-1 (control) strain and the 2× Protease-1 (wyD overexpressed; PspoVG-yvyD) strain at time points: twelve (12), twenty (20), thirty-six (36), forty-five (45), sixty-one (61), sixty-eight (68), seventy-three (73) and eighty-four (84) hours; and a protease activity assay performed to determine the effect of yvyD overexpression from the spoVG promoter (PspoVG) on the production of Protease-1. The results of the protease assay (FIG. 2), demonstrate that there is a trend towards increased Protease-1 production at the end of fermentation, and significant enhancement in production at 68 hours to about 73 hours, due to yvyD overexpression.

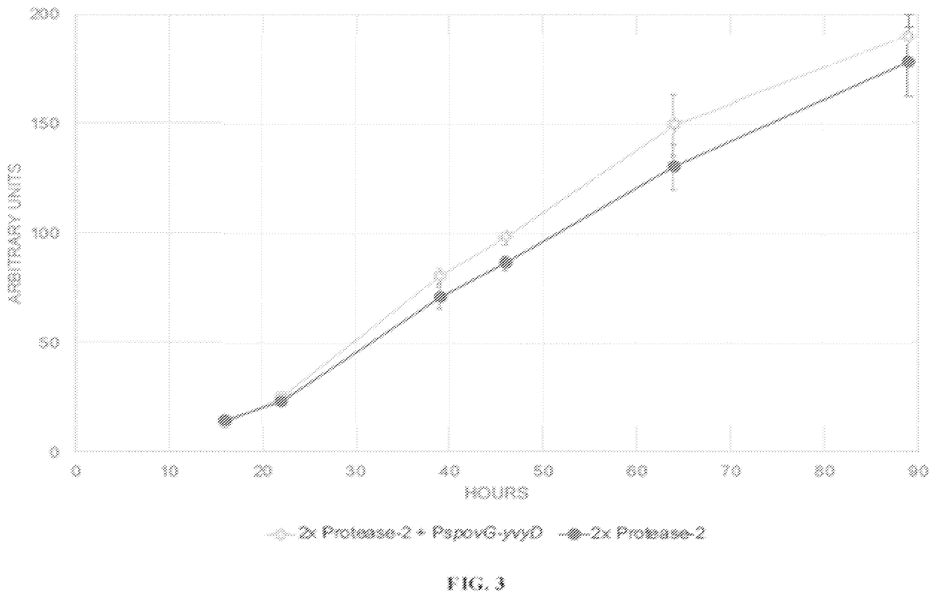

Likewise, Example 3 assessed the PspoVG-yvyD cassette for increased yvyD expression via reporter Protease-2 production in the B. subtilis strains described in Example 2. More particularly, sample aliquots were taken from the 2× Protease-2 (control) strain and the 2× Protease-2 (yvyD overexpressed; PspoVG-yvyD) strain at time points: sixteen (16), twenty-two (22), thirty-nine (39), forty-six (46), sixty-four (64) and eighty-nine (89) hours; and a protease activity assay performed to determine the effect of yvyD overexpression from the spoVG promoter (PspoVG) on the production of Protease-2. The results of the protease assay (FIG. 3) demonstrate a trend towards increased Protease-2 production starting at about 39 hours until the end of fermentation, and significant enhancement in production of Protease-2 at 39 and 46 hours, due to yvyD overexpression.

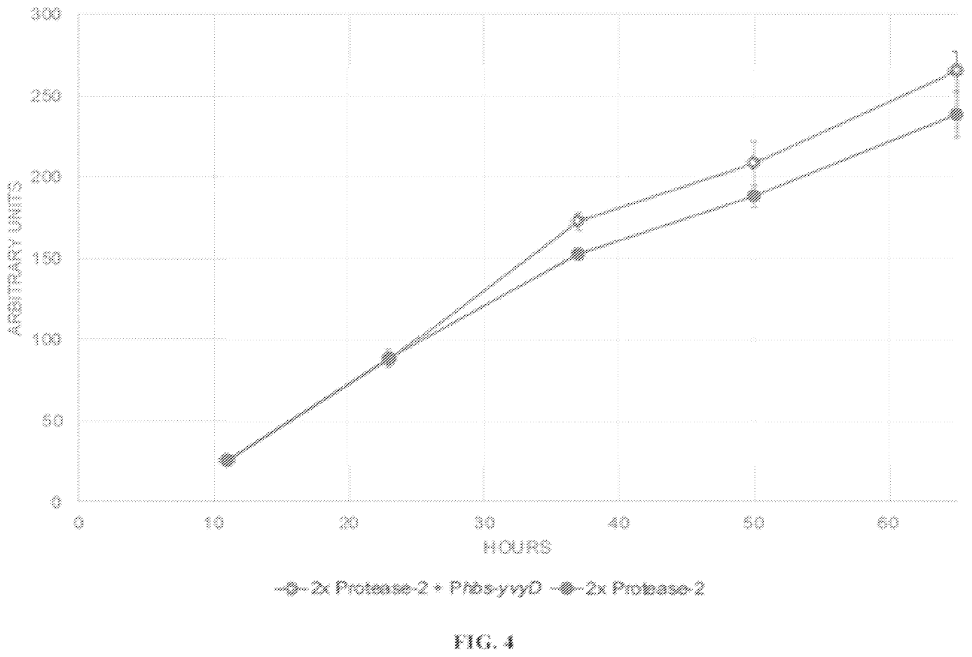

As further described in Example 3, Applicant assessed the Phbs-yvyD cassette for increased yvyD expression via reporter Protease-2 production in the B. subtilis strains described in Example 2. More particularly, sample aliquots were taken from the 2× Protease-2 (control) strain and the 2× Protease-2 (yvyD overexpressed; Phbs-yvyD) strain at time points: eleven (11), twenty-three (23), thirty-seven (37), fifty (50) and sixty-five (65) hours; and a protease activity assay performed to determine the effect of yvyD overexpression from the Phbs promoter (Phbs) on the production of Protease-2. The results of the protease assay (FIG. 4), demonstrate a trend towards increased Protease-2 production starting at about thirty-seven (37) hours until the end of fermentation, and a significant enhancement in production of Protease-2 at 37 hours, due to yvyD overexpression.

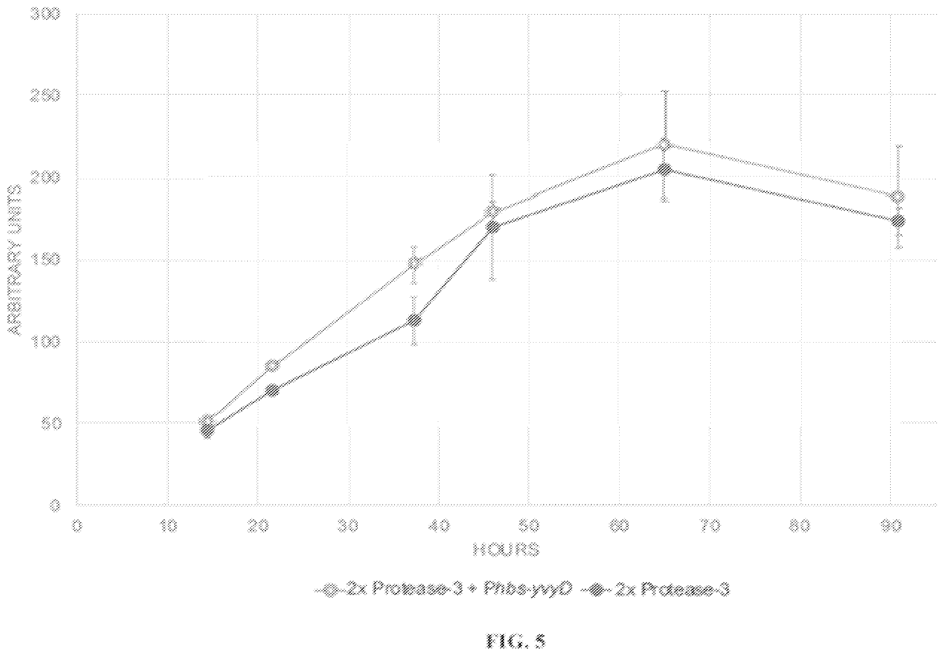

As further presented in Example 3, Applicant assessed the Phbs-yvyD cassette for increased yvyD expression via reporter Protease-3 production in the B. subtilis strains described in Example 2. More particularly, sample aliquots were taken from the 2× Protease-3 (control) strain and the 2× Protease-3 (yvyD overexpression; Phbs-yvyD) strain at time points fourteen (14), twenty-two (22), thirty-seven (37), forty-six (46), sixty-five (65) expression from the Phbs promoter (Phbs) on the production of Protease-3. The results of the protease assay (FIG. 5), demonstrate a trend towards increased Protease-3 production starting at about 22 hours until the end of fermentation, and a significant enhancement in production of Protease-3 at 22 hours and 37 hours, due to yvyD overexpression.

Thus, as described herein, certain embodiments of the disclosure are related to the surprising and unexpected observation that overexpression of the yvyD gene CDS results in enhanced production of proteins of interest in Gram-positive bacterial cells. Certain aspects of the disclosure are therefore related to recombinant Gram-positive bacterial cells/strains expressing a yvyD gene CDS from a promoter that produces higher steady-state levels of mRNA than the native yvyD promoter. For example, as generally described by Zhu and Stülke (2017), the steady-state mRNA levels of spoVG expressed from its native promoter are higher than steady-state yvyD mRNA levels.

In particular, the results from the Zhu and Stülke study (2017) performed under four different growth conditions are reproduced below in TABLE 1, wherein the Conditions, exponential growth+glucose (labeled “LBGexp”), fermentation (labeled “Ferm”); time before glucose exhaustion (labeled “T”) and exponential growth (labeled “LBexp”), are presented in the first column (TABLE 1) and the steady-state mRNA levels of yvyD, spoVG and hbs under the specified conditions are presented in columns 2-4 (TABLE 1), respectively. More particularly, the steady-state levels of spoVG mRNA are higher than yvyD (hpf) mRNA levels in more than 80% of transcriptome data points from fifty-three (53) experimental conditions (Zhu and Stülke, 2017). Importantly, growth conditions that are most relevant to industrial fermentation of Gram-positive strains demonstrate that spoVG steady-state mRNA levels are higher than yvyD (TABLE 1).

TABLE 1
Steady-State Levels of hpf (yvyD), spoVG and hbs mRNA
Determined in Four Different Growth Conditions (Data
reproduced from Zhu and Stülke, 2017)

| Condition | hpf (yvyD) | spoVG | hbs |
|---|---|---|---|
| LBGexp | 11.5 | 12.97 | 16.45 |
| Ferm | 15.02 | 16.28 | 15.95 |
| T-5.40 h | 14.66 | 15.8 | 16.17 |
| T-4.40 h | 14.66 | 16.21 | 16.24 |
| T-3.40 h | 14.58 | 16.34 | 16.31 |
| T-2.40 h | 14.47 | 15.92 | 16.17 |
| T-1.40 h | 14.43 | 16.09 | 16.07 |
| T-1.10 h | 14.52 | 15.52 | 15.69 |
| T-0.40 h | 14.44 | 15.95 | 16.07 |
| T-0.0 h | 14.26 | 15.61 | 15.83 |
| LBexp | 13.81 | 14.17 | 16.46 |

As indicated in TABLE 1, a similar trend was observed for hbs steady-state mRNA levels relative to yvyD (hpf), wherein more than 85% of hbs mRNA transcriptome data points from 53 experimental conditions are higher than yvyD steady-state mRNA levels. (Zhu and Stulke, 2017). Thus, as summarized in the data reproduced in TABLE 1, steady-state levels of hbs mRNA are higher than yvyD levels under the LBGexp, Ferm time before glucose exhaustion (T) and LBexp growth conditions.

More particularly, as described in the Examples below, Applicant has experimentally demonstrated that the overexpression of yvyD in recombinant Gram-positive bacterial cells increases the amount of three different reporter proteins produced (e.g., see FIG. 2-FIG. 5). For example, the effect of increased steady-state yvyD mRNA on the production of proteins has been demonstrated for two (2) different yvyD overexpression cassettes, which use promoter swap mutations (PspoVG-yvyD; Phbs-yvyD) that increase the relative amount of steady state mRNA (TABLE 1). Likewise, as described and contemplated herein, Applicant expects that other means to increase yvyD steady-state mRNA levels will have similarly beneficial effects on the production of proteins of interest.

For example, other possible means to increase yvyD mRNA levels include, but are not limited to, yvyD overexpression cassettes that use promoters from other genes to increase yvyD steady-state mRNA levels above native yvyD steady-state mRNA levels, yvyD overexpression cassettes that use non-Bacillus subtilis heterologous promoters to increase yvyD steady-state mRNA levels above native yvyD steady-state mRNA levels, plasmid-based expression cassettes of yvyD from its native promoter (PyvyD-yvyD), integration of multiple copies of PyvyD-yvyD into the genome, relocation of the yvyD locus to a genomic region that increases yvyD expression, host modifications that increase yvyD mRNA steady-state levels (e.g., mRNA degradation pathways), mutations within transcribed yvyD that affect mRNA stability and the like. Furthermore, mutations within transcribed yvyD that affect mRNA translation (e.g., more efficient ribosomal binding sites) are expected to increase YvyD levels within the cell and increase the production of proteins of interest.

Thus, without wishing to be bound by any theory, mechanism, or mode of action, Applicant contemplates herein that increased YvyD protein levels enhance the production of target proteins of interest. More specifically, as demonstrated in the Examples, recombinant Gram-positive cells expressing increased levels of YvyD produce increased amounts of reporter proteins (compared to control cells), demonstrating increased specific productivities (Qp) and increased carbon efficiency of the reporter proteins produced when cultivated under suitable conditions. In certain one or more aspects or embodiments, the enhanced production of proteins of interest described in the Examples (FIG. 2-FIG. 5) may be explained by at least two possible mechanisms due to increased yvyD expression. In a first possible mechanism, YvyD promotes the stability of ribosomal associated proteins (Feaga et al., 2020) via the YvyD ribosome dimerization function. In a second possible mechanism, the pool of free ribosomes are reduced by (YvyD) ribosome dimerization (e.g., via higher YvyD levels than normal (native) YvyD levels, which promotes the translation of highly expressed gene of interest (GOI) mRNAs and/or GOI mRNAs with efficient ribosome binding sites.

In certain other one or more aspects or embodiments, a Gram-positive bacterial wvyD gene comprises sequence homology to the B. subtilis yvyD gene of SEQ ID NO: 23. In certain other embodiments, an overexpressed yvyD gene comprises sequence homology to the B. subtilis yvyD gene of SEQ ID NO: 23 (e.g., comprising at least about 50% sequence identity to SEQ ID NO: 23) and encodes a functional YvyD protein. In other embodiments, an overexpressed yvyD gene encodes a YvyD protein comprising sequence homology to the native B. subtilis YvyD protein of SEQ ID NO: 26. In certain embodiments, an overexpressed yvyD gene encodes a functional YvyD protein comprising at least about 50% sequence identity to SEQ ID NO: 26.

In related aspects, a Gram-positive bacterial yvyD gene (or yvyD gene homologue) encodes a functional “general stress factor protein” (or “ribosome hibernation promotion factor”) comprising sequence homology to the YvyD protein of SEQ ID NO: 26 (or a YvyD homologue thereof). For example, as briefly set forth above, the mechanisms for ribosome maintenance in dormant bacteria have been characterized in certain bacteria (Franklin et al., 2020), and include “ribosomal accessory proteins” such as ribosome modulation factor (RMF), hibernation-promoting factor (HPF), and the HPF paralog (YfiA), wherein structures of ribosomes with HPF and/or RMF in their active sites have been solved for several diverse species of bacteria.

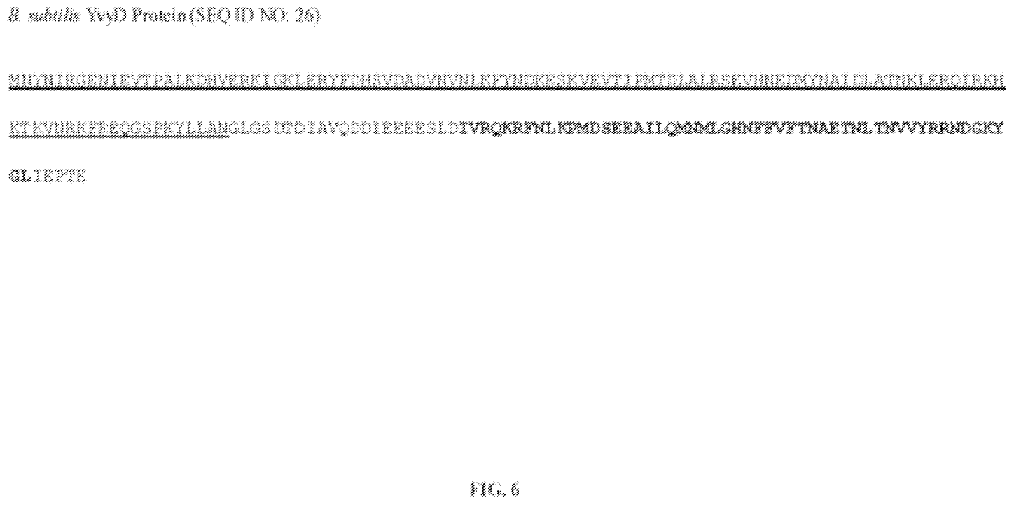

In certain embodiments, Gram-positive bacterial yvyD genes (homologues) may be identified via sequence alignments. For example, the native B. subtilis YvyD protein (amino acid) sequence is shown in FIG. 6 (SEQ ID NO: 26), wherein the full length protein sequence comprises a conserved N-terminal domain (underlined residues; RaiA superfamily domain) and a conserved C-terminal domain (bold residues; Ribosome S30AE_C superfamily domain). In particular, the conserved N-terminal domain RaiA superfamily domain present in the B. subtilis YvyD protein (SEQ ID NO: 26) is set forth in SEQ ID NO: 27; and the conserved C-terminal domain Ribosome S30AE_C superfamily domain present in the B. subtilis YvyD protein (SEQ ID NO: 26) is set forth in SEQ ID NO: 28.

For example, the “ribosome-associated inhibitor A” (RaiA) protein is known as a stress-response protein that binds the ribosomal subunit interface and arrests translation by interfering with aminoacyl-tRNA binding to the ribosomal A site, wherein the RaiA fold structurally resembles the double-stranded RNA-binding domain (dsRBD). Likewise, the Ribosome S30AE_C superfamily domain often occurs at the C-terminus of ribosomal stress response proteins (e.g., Sigma 54 modulation/S30EA ribosomal proteins).

Thus, in certain aspects, a Gram-positive bacterial yvyD gene encodes a YvyD protein comprising at least about 50%-100% identity to the B. subtilis N-terminal RaiA superfamily domain of SEQ ID NO: 27. In other embodiments, a Gram-positive bacterial yvyD gene encodes a YvyD protein comprising at least about 50%-100% identity to the B. subtilis C-terminal Ribosome S30AE_C superfamily domain of SEQ ID NO: 28. In certain other embodiments, a Gram-positive bacterial yvyD gene encodes a YvyD protein comprising at least about 50%-100% identity to SEQ ID NO: 27 and at least about 50%-100% identity to SEQ ID NO: 28.

### III. Microbial Host Cells

As briefly stated above, certain embodiments are related to recombinant microbial (host) cells expressing genes encoding proteins of interest and the like. In certain aspects Gram-positive bacterial cells (strains) include the classes Bacilli, Clostridia and Mollicutes (e.g., including Lactobacillus with the families Aerococcaceae, Carnobacteriaceae, Enterococcaceae, Lactobacillus, Leuconostocaceae, Oscillospiraceae, Streptococcaceae and the Bacillales with the families Alicyclobacellaceae, Bacillaceae, Caryophanaceae, Listeriaceae, Paenibacillaceae, Planococcaceae, Sporolactobacillus, Staphylococcaceae, Thermoactinomycetaceae, Turicibacteraceae). In certain aspects, Gram-positive bacterial cells (strains) are Streptomyces.

Species of the family Bacillaceae include Alkalibacillus, Amphibacillus, Anoxybacillus, Bacillus, Caldalkalibacillus, Cerasilbacillus, Exiguobacterium, Filobacillus, Geobacillus, Gracilibacillus, Halobacillus, Halolactibacillus, Jeotgalibacillus, Lentibacillus, Marinibacillus, Oceanobacillus, Ornithinibacillus, Paraliobacillus, Paucisalibacillus, Pontibacillus, Pontibacillus, Saccharococcus, Salibacillus, Salinibacillus, Tenuibacillus, Thalassobacillus, Ureibacillus, Virgibacillus.

As used herein, “the genus Bacillus” includes all species within the genus “Bacillus” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”.

In certain aspects, Bacillus sp. cells include, but are not limited to, B. acidiceler, B. acidicola, B. acidocaldarius, B. acidoterrestris, B. aeolius, B. aerius, B. aerophilus, B. agaradhaerens. B. agri, B. aidingensis, B. akibai, B. alcalophilus, B. algicola, B. alginolyticus, B. alkalidiazo-trophicus, B. alkalinitrilicus, B. alkalitelluris, B. altitudinis, B. alveayuensis, B. alvei, B. amylolyticus, B. aneurinilyticus, B. aneurinolyticus, B. anthracia, B. aquimaris, B. arenosi, B. arseniciselenatis, B. arsenicoselenatis, B. arsenicus, B. arvi, B. asahii, B. atrophaeus, B. aurantiacus, B. axarquiensis, B. azotofixans, B. azotoformans, B. badius, B. barbaricus, B. bataviensis, B. beijingensis, B. benzoevorans, B. bogoriensis, B. boroniphilus, B. borstelenis, B. butanolivorans, B. carboniphilus, B. cecembensis, B. cellulosilyticus, B. centrosporus, B. chagannorensis, B. chitinolyticus, B. chondroitinus, B. choshinensis, B. cibi, B. circulans, B. clarkii, B. clausii, B. coagulans, B. coahuilensis, B. cohnii, B. curdianolyticus, B. cycloheptanicus, B. decisifrondis, B. decolorationis, B. dipsosauri, B. drentensis, B. edaphicus, B. ehimensis, B. endophyticus, B. farraginis, B. fastidiosus, B. firmus, B. plexus, B. foraminis, B. fordii, B. formosus, B. fortis, B. fumarioli, B. funiculus, B. fusiformis, B. galactophilus, B. galactosidilyticus, B. gelatini, B. gibsonii, B. ginsengi, B. ginsengihumi, B. globisporus, B. globisporus subsp. globisporus, B. globisporus subsp. marinus, B. glucanolyticus, B. gordonae, B. halmapalus, B. haloalkaliphilus, B. halodenitrificans, B. halodurans, B. halophilus, B. hemicellulosilyticus, B. herbersteinensis, B. horikoshii, B. horti, B. hemi, B. hwajinpoensis, B. idriensis, B. indicus, B. infantis, B. infernus, B. insolitus, B. isabeliae, B. jeotgali, B. kaustophilus, B. kobensis, B. koreensis, B. kribbensis, B krulwichiae, B. laevolacticus, B. larvae, B. laterosporus, B. lautus, B. lehensis, B. lentimorbus, B. lentus, B. litoralis, B. luciferensis, B. macauensis, B. macerans, B. macquariensis, B. macyae, B. malacitensis, B. mannanilyticus, B. marinus, B. marisflavi, B. marismortui, B. massiliensis, B. methanolicus, B. migulanus, B. mojavensis, B. mucilaginosus, B. muralis, B. murimartini, B. mycoides, B. naganoensis, B. nealsonii, B. neidei. B, niabensis, B. niacini, B. novalis, B. odysseyi, B. okhensis, B. okuhidensis, B. oleronius, B. oshimensis, B. pabuli, B. pallidus, B. pallidus (illeg.), B. panaciterrae, B. pantothenticus, B. parabrevis, B. pasteurii, B. patagoniensis, B. peoriae, B. plakortidis, B. pocheonensis, B. polygoni, B. polymyxa, B. popilliae, B. pseudalcaliphilus, B. pseudofirmus, B. pseudomycoides, B. psychrodurans, B. psychrophilus, B. psychrosaccarolyticus, B. psychrotolerans, B. pulvifaciens, B. pycnus, B. qingdaonensis, B. reuszeri, B. runs, B. safensis, B. salarius, B. salexigens, B. saliphilus, B. schlegelii, B. selenatarsenatis, B. selenitrireducens, B. seohaeanensis, B. shackletonii, B. silvestris, B. simplex, B. siralis, B. smithii, B. soli, B. sonorensis, B. sphaericus, B. sporothermodurans, B. stearothermophilus, B. stratosphericus, B. subterraneus, B. subtilis subsp. spizizenii, B. subtilis subsp. subtilis, B. taeanensis, B. tequilensis, B. thermantarcticus, B. thermoaerophilus, B. thermoamylovorans, B. thermoantarcticus, B. thermocatenulatus, B. thermocloacae, B. thermodenitrificans, B. thermoglucosidasius, B. thermoleovorans, B. thermoruber, B. thermosphaericus, B. thiaminolyticus, B. thioparans, B. thuringiensis, B. tusciae, B. validus, B. vallismortis, B. vedderi, B. velezensis, B. vietnamensis, B. vireti, B. vulcani, B. wakoensis and B. weihenstephanensis.

### IV. Recombinant Polynucleotides and Molecular Biology

Suitable nucleic acid (DNA) control sequences, regulatory sequences and the like for constructing yvyD overexpressed polynucleotide cassettes include promoter sequences and functional parts thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like. In particular embodiments, promoter region sequences are generally chosen so that they are functional in the Gram-positive bacterial cells and overexpress a yvyD gene CDS relative to the expression of the yvyD gene CDS from its wild-type yvyD promoter region (SEQ ID NO: 24).

For example, promoters useful for driving gene expression in Bacillus cells include, but are not limited to, the B. subtilis alkaline protease (aprE) promoter, the α-amylase promoter (amyE) of B. subtilis, the α-amylase promoter (amyL) of B. licheniformis, the α-amylase promoter of B. amyloliquefaciens, the neutral protease (nprE) promoter from B. subtilis, a mutant aprE promoter, or any other promoter from B licheniformis or other related Bacilli. Methods for screening and creating promoter libraries with a range of activities (promoter strength) in Bacillus cells is describe in Publication No. WO2002/14490.

An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

Bacillus host cells. Certain aspects are therefore related to polynucleotides (e.g., expression cassettes) comprising an upstream (5′) promoter (pro) sequence operably linked to a downstream nucleic acid sequence (ss) encoding a modified (protein) signal sequence operably linked to a downstream (3′) nucleic acid sequence (poi) encoding a protein of interest.

Certain embodiments of the disclosure are directed to isolated nucleic acids (polynucleotides). Thus, certain aspects are related to plasmids, vectors, expression cassettes and the like comprising a polynucleotide sequence encoding proteins of the disclosure. Likewise, other embodiments are directed to recombinant microbial cells (strains) expressing one or more heterologous proteins. More particularly, in certain embodiments, a gene, polynucleotide, open reading frame and the like of the disclosure are genetically modified. In certain aspects, genetic modifications include, but are not limited to, (a) the introduction, substitution, or removal of one or more nucleotides in a gene, a gene coding sequence (CDS), an open reading frame (ORF) or the introduction, substitution, or removal of one or more nucleotides in a regulatory element required for the transcription or translation of the gene (or gene CDS), (b) a gene disruption, (c) a gene conversion, (d) a gene deletion, (e) the down-regulation of a gene (e.g., interfering RNA), (f) specific mutagenesis and/or (g) random mutagenesis of any one or more the genes or polynucleotides disclosed herein.

Those of skill in the art are well aware of suitable methods for introducing polynucleotide sequences into bacterial cells (e.g., E. coli, Bacillus sp., etc.), filamentous fungal cells (e.g., Aspergillus sp., Trichoderma sp., etc.), yeast cells (e.g., Saccharomyces sp.) and the like (i.e., microbial cells).

As generally specified above, certain embodiments of the disclosure are directed to expressing, producing and/or secreting one or more proteins of interest which are heterologous to the to the microbial host cell. Therefore, the instant disclosure generally relies on routine techniques in the field of recombinant genetics. Basic texts disclosing the general methods of use in present disclosure include Sambrook et al., (1989; 2011; 2012); Kriegler (1990) and Ausubel et al., (1987; 1994).

In particular embodiments, the disclosure relates to recombinant (modified) nucleic acids comprising a gene CDS encoding a YvyD protein. For example, in certain aspects, a recombinant nucleic acid is a polynucleotide expression cassette suitable for expression of a YvyD protein.

In certain other embodiments, recombinant nucleic acids (polynucleotides) comprise one or more selectable markers. Selectable markers for use in Gram-negative bacteria, Gram-positive bacteria, filamentous fungi and yeast are generally known in the art. Thus, in certain embodiments, a polynucleotide construct encoding YvyD protein and/or a polynucleotide construct encoding a protein of interest (POI) comprises a nucleic acid sequence encoding a selectable marker operably linked thereto.

In other embodiments, nucleic acids comprising a gene or gene CDS encoding YvyD protein further comprise operably linked regulatory or control sequences. An example of regulatory or control sequences may be a promoter sequence or a functional part thereof. (i.e., a part which is sufficient for affecting expression of the nucleic acid sequence). Other control sequences include, but are not limited to, a leader sequence, a pro-peptide sequence, a signal sequence, a transcription terminator, a transcriptional activator and the like. Thus, in certain embodiments, a recombinant (modified) polynucleotide comprises an upstream (5′) promoter (pro) sequence driving the expression of a gene coding sequences (CDS) encoding a YvyD protein, or a POI of the disclosure. More particularly, in certain embodiments, the promoter is a constitutive or an inducible promoter active (functional) in the microbial host cell. For example, one of sill in the art can use any suitable promoter capable of driving the expression of a gene of interest in a microbial expression host cell. Thus, in certain aspects, a recombinant nucleic acid of the disclosure comprises a promoter (pro) sequence which is 5′ (upstream) and operably linked to a nucleic acid sequence (gene CDS) encoding a YvyD protein (e.g., 5′-[pro]-[gene CDS]-3′).

In certain other aspects, a recombinant nucleic acid (e.g., an expression cassette) comprises an upstream (5′) promoter (pro) sequence operably linked to a downstream (3′) nucleic acid sequence (gene CDS) encoding a YvyD protein (or encoding a POI), further comprises a terminator (term) sequence downstream and operably linked thereto. For example, in certain aspects, a recombinant nucleic acid of the disclosure comprises a promoter (pro) sequence which is 5′ (upstream) and operably linked to a nucleic acid sequence (gene CDS) encoding a YvyD protein (or a POI) which is operably linked to a downstream terminator (term) sequence (e.g., 5′-[pro]-[gene CDS]-[term]-3′).

Suitable promoters for driving the expression of genes of interest in a microbial host cell of the disclosure are generally known in the art. For example, exemplary Bacillus sp. promoters include, but are not limited to, tac promoter sequences, β-lactamase promoter sequences, aprE promoter sequences, groES promoter sequences, ftsH promoter sequences, tufA promoter sequences, secDF promoter sequences, minC promoter sequences, spoVG promoter sequences, veg promoter sequences, hbs promoter sequences, amylases promoter sequences, P43 promoter sequence and the like. exemplary filamentous fungal promoters include, but are not limited to, Trichoderma sp. promoters (e.g., cellobiohydrolase promoters, endoglucanase promoters, β-glucosidase promoters, xylanases promoters, glucoamylase promoters), Aspergillus sp. promoters (e.g., trpC promoters, glucoamylase promoters), and the like. However, it is not intended that the present disclosure be limited to any particular promoter, as any suitable promoter known to those in the art finds use with the present invention.

Thus, certain other embodiments are related to cultivating (fermenting) microbial host cells expressing a POI, wherein the expressed POI is secreted into the culture (fermentation) broth. For example, in certain other embodiments, a recombinant nucleic acid comprises an upstream (5′) heterologous promoter (pro) sequence operably linked to a downstream (3′) nucleic acid sequence (ss) encoding a protein signal sequence operably linked to a downstream (3′) nucleic acid sequence (GOI) encoding a protein of interest (e.g., 5′-[pro]-[ss]-[GOI]-3′).

Any suitable (protein) signal sequence (signal peptide) functional in the microbial cell of choice may be used for the secretion (transport) of mature proteins of interest. The signal sequence is typically located N-terminal to the precursor or mature protein sequence. For example, suitable signal sequences for use include, but are not limited to, signal sequences from secreted proteases, peptidases, amylases, glucoamylases, cellulases, lipases, esterases, arabinases, glucanases, chitosanases, lyases, xylanases, nucleases, phosphatases, transport and binding proteins, etc. In certain embodiments, a signal sequence is selected from an aprE signal sequence, a nprE signal sequence, a vpr signal sequence, a bglC signal sequence, a bglS signal sequence, a sacB signal sequence and amylase signal sequence, a heterologous signal sequence and/or a synthetic signal sequence,

Thus, in certain embodiments, standard techniques for transformation of microbial cells (which are well known to one skilled in the art) are used to transform a microbial host cell of the disclosure. Thus, the introduction of a DNA construct or vector into a host cell includes techniques such as transformation, electroporation, nuclear microinjection, transduction, transfection (e.g., lipofection mediated and DEAE-Dextrin mediated transfection), incubation with calcium phosphate DNA precipitate, high velocity bombardment with DNA-coated microprojectiles, gene gun or biolistic transformation, protoplast fusion and the like. General transformation techniques are known in the art.

In certain embodiments, a heterologous gene, polynucleotide or ORF is cloned into an intermediate vector, before being transformed into a the microbial (host) cells for replication and/or expression. These intermediate vectors can be prokaryotic vectors, such as, e.g., plasmids, or shuttle vectors. Thus, the expression vector/construct typically contains a transcription unit or expression cassette that contains all the additional elements required for the expression of the heterologous sequence. For example, a typical expression cassette contains a 5′ promoter operably linked to the heterologous nucleic acid sequence encoding a protein of interest and may further comprise sequence signals required for efficient polyadenylation of the transcript, ribosome binding sites, and translation termination. Additional elements of the cassette may include enhancers and, if genomic DNA is used as the structural gene, introns with functional splice donor and acceptor sites.

The particular expression vector used to transport the genetic information into the cell is not particularly critical. Any of the conventional vectors used for expression in eukaryotic or prokaryotic cells may be used. Standard bacterial expression vectors include bacteriophages 2 and M13, as well as plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. The elements that can be included in expression vectors may also be a replicon, a gene encoding antibiotic resistance to permit selection of bacteria that harbor recombinant plasmids, or unique restriction sites in nonessential regions of the plasmid to allow insertion of heterologous sequences.

The methods of transformation of the present invention may result in the stable integration of all or part of the transformation vector into the genome of the microbial cell. However, transformation resulting in the maintenance of a self-replicating extra-chromosomal transformation vector is also contemplated. Any of the known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other known methods for introducing cloned genomic DNA, cDNA, synthetic DNA or other foreign genetic material into a host cell (see, e.g., Sambrook et al., supra). Also of use are Agrobacterium-mediated transfection methods. It is only necessary that the particular genetic engineering procedure used be capable of successfully introducing at least one gene into the host cell capable of expressing the heterologous gene.

After the expression vector is introduced into the cells, the transfected cells are cultured under conditions favoring expression of the genes of interest. Large batches of transformed cells can be cultured as described herein. Finally, the broth and/or product(s) are recovered from the culture using standard techniques. Thus, the disclosure herein provides for the expression and secretion of desired proteins.

Microbial cells of the disclosure may comprise genetic modifications of one or more endogenous genes and/or one or more introduced (heterologous) genes described herein. For example, microbial cells may be constructed to reduce or eliminate the expression of endogenous genes (e.g., reduce or eliminate genes encoding proteases), using methods well known in the art, e.g., insertions, disruptions, replacements, or deletions. The portion of the gene to be modified or inactivated may be, for example, the coding region or a regulatory element required for expression of the coding region.

In certain embodiments, a modified cell of the disclosure is constructed by introducing, substituting, or removing one or more nucleotides in the gene or a regulatory element required for the transcription or translation thereof. For example, nucleotides may be inserted or removed so as to result in the introduction of a stop codon, the removal of the start codon, or a frame-shift of the open reading frame. Such a modification may be accomplished by site-directed mutagenesis or PCR generated mutagenesis in accordance with methods known in the art.

In another embodiment, a modified cell is constructed by the process of gene conversion. For example, in the gene conversion method, a nucleic acid sequence corresponding to the gene(s) is mutagenized in vitro to produce a defective nucleic acid sequence, which is then transformed into the parental cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene. Alternatively, the defective nucleic acid sequence may contain an insertion, substitution, or deletion of one or more nucleotides of the gene, as described below.

In other embodiments, a modified cell is constructed by established anti-sense techniques using a nucleotide sequence complementary to the nucleic acid sequence of the gene. More specifically, expression of the gene by a cell may be reduced (down-regulated) or eliminated by introducing a nucleotide sequence complementary to the nucleic acid sequence of the gene, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other embodiments, a modified cell is produced/constructed via CRISPR-Cas9 editing. For example, a gene of interest can be disrupted (or deleted or down-regulated) by means of nucleic acid guided endonucleases, that find their target DNA by binding either a guide RNA (e.g., Cas9) and Cpf1 or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the microbial cell and a terminator active in the microbial cell, thereby creating a microbial cell Cas9 expression cassette. Likewise, one or more target sites unique to the gene of interest are readily identified by a person skilled in the art. For example, to build a DNA construct encoding a gRNA-directed to a target site within the gene of interest, the variable targeting domain (VT) will comprise nucleotides of the target site which are 5′ of the (PAM) protospacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a microbial cell expression cassette for the gRNA is created by operably linking the DNA encoding the gRNA to a promoter active in the microbial cells and a terminator active in the microbial cells. The Cas9 expression cassette, the gRNA expression cassette and the editing template can be co-delivered to cells using many different methods (e.g., protoplast fusion, electroporation, natural competence, or induced competence). The transformed cells are screened by PCR amplifying the target gene locus, by amplifying the locus with a forward and reverse primer. These primers can amplify the wild-type locus or the modified locus that has been edited by the RGEN.

In yet other embodiments, a modified cell is constructed by random or specific mutagenesis using methods well known in the art, including, but not limited to, chemical mutagenesis and transposition. Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods. Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

### V. Fermenting Gram-Positive Cells for Production of Proteins of Interest

In certain embodiments, the present disclosure provides recombinant microbial cells capable of producing proteins of interest. More particularly, certain embodiments are related genetically modified microbial cells expressing heterologous polynucleotides encoding proteins of interest, genetically microbial cells co-expressing heterologous proteins of interest and a YvyD protein, and the like. Thus, particular embodiments are related to cultivating (fermenting) microbial cells for the production of proteins of interest.

In general, fermentation methods well known in the art are used to ferment the microbial cells. In some embodiments, the cells are grown under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within batch cultures, cells progress through a static lag phase to a high growth log phase and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO2. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Culturing/fermenting is generally accomplished in a growth medium comprising an aqueous mineral salts medium, organic growth factors, a carbon and energy source material, molecular oxygen, and, of course, a starting inoculum of the microbial host to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and also present preferably should be certain trace elements such as copper, manganese, molybdenum, zinc, iron, boron, and iodine, and others, again in suitable soluble assimilable form, all as known in the art.

The fermentation reaction is an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The fermentation temperature can vary somewhat, but for most microbial cells the temperature generally will be within the range of about 20° C. to 40° C.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations, and can be employed by bubbling through the aqueous ferment (fermentation medium) in suitable amounts. At the same time, such ammonia can also be employed to assist in pH control.

The pH range in the aqueous microbial ferment (fermentation admixture) should be in the exemplary range of about 2.0 to 8.0. Preferences for pH range of microorganisms are dependent on the media employed to some extent, as well as the particular microorganism, and thus change somewhat with change in media as can be readily determined by those skilled in the art.

Preferably, the fermentation is conducted in such a manner that the carbon-containing substrate can be controlled as a limiting factor, thereby providing good conversion of the carbon-containing substrate to cells and avoiding contamination of the cells with a substantial amount of unconverted substrate. The latter is not a problem with water-soluble substrates, since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermenter, cell density measurable by dry cell weights, light transmittancy, or the like. The feed rates of the various materials can be varied so as to obtain as rapid a cell growth rate as possible, consistent with efficient utilization of the carbon and energy source, to obtain as high a yield of microorganism cells relative to substrate charge as possible.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

### VI. Proteins of Interest

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest. Thus, in certain embodiments, a modified cell of the disclosure expresses an endogenous POI, a heterologous POI, or a combination of one or more of such POIs.

In certain embodiments, a modified cell may produce an increased amount of a POI (e.g., protein having DNase activity) relative to a parental (control) cell, wherein the increased amount of the POI is at least about a 0.01% increase, at least about a 0.10% increase, at least about a 0.50% increase, at least about a 1.0% increase, at least about a 2.0% increase, at least about a 3.0% increase, at least about a 4.0% increase, at least about a 5.0% increase, or an increase greater than 5.0%. In certain embodiments, the increased amount of the POI is determined by assaying enzymatic activity and/or by assaying/quantifying the specific productivity (Qp) thereof. Likewise, one skilled in the art may utilize other routine methods and techniques known in the art for detecting, assaying, measuring, etc. the expression, production or secretion of one or more proteins of interest.

In certain embodiments, a POI or a variant POI thereof is selected from the group consisting of acetyl esterases, aminopeptidases, amylases, arabinases, arabinofuranosidases, arylesterases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, lysozymes, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phosphodiesterases, phytases, polyesterases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1, EC 2, EC 3, EC 4, EC 5 or EC 6.

For example, in certain embodiments a POI is an enzyme selected from an oxidoreductase (EC 1), a transferase (EC 2), a hydrolase (EC 3), a lyase (EC 4) and an isomerase (EC 5).

Thus, in certain embodiments, industrial protease producing Gram-positive host cells provide particularly useful expression hosts. Likewise, in certain other embodiments, industrial amylase producing Gram-positive host cells provide particularly useful expression hosts. For example, there are two general types of proteases which are typically secreted by Bacillus sp., namely neutral (or “metalloproteases”) and alkaline (or “serine”) proteases. For example, Bacillus subtilisin proteins (enzymes) are exemplary serine proteases for use in the present disclosure. A wide variety of Bacillus subtilisins have been identified and sequenced, for example, subtilisin 168, subtilisin BPN′, subtilisin Carlsberg, subtilisin DY, subtilisin 147 and subtilisin 309. In some embodiments, modified Gram-positive cells produce mutant (i.e., variant) proteases. Thus, in certain embodiments, modified (recombinant) Gram-positive cells comprise expression constructs encoding native and/or variant proteases.

In certain other embodiments, modified Gram-positive cells comprises an expression construct encoding an amylase. A wide variety of amylase enzymes and variants thereof are known to one skilled in the art. Thus, in certain embodiments, modified (recombinant) Gram-positive cells comprise expression constructs encoding native and/or variant proteases.

In other embodiments, a POI or variant POI expressed and produced in a modified cell of the disclosure is a peptide, a peptide hormone, a growth factor, a clotting factor, a chemokine, a cytokine, a lymphokine, an antibody, a receptor, an adhesion molecule, a microbial antigen (e.g., HBV surface antigen, HPV E7, etc.), variants thereof, fragments thereof and the like. Other types of proteins (or variants thereof) of interest may be those that are capable of providing nutritional value to a food or to a crop. Non-limiting examples include plant proteins that can inhibit the formation of anti-nutritive factors and plant proteins that have a more desirable amino acid composition (e.g., a higher lysine content than a non-transgenic plant).

There are various assays known to those of ordinary skill in the art for detecting and measuring activity of intracellularly and extracellularly expressed proteins. In particular, for proteases, there are assays based on the release of acid-soluble peptides from casein or hemoglobin measured as absorbance at 280 nm or colorimetrically, using the Folin method. Other exemplary assays include succinyl-Ala-Ala-Pro-Phe-para-nitroanilide assay (SAAPFpNA) and the 2,4,6-trinitrobenzene sulfonate sodium salt assay (TNBS assay).

International PCT Publication No. WO2014/164777 discloses Ceralpha α-amylase activity assays useful for amylase activities described herein.

Means for determining the levels of secretion of a protein of interest in a host cell and detecting expressed proteins include the use of immunoassays with either polyclonal or monoclonal antibodies specific for the protein. Examples include enzyme-linked immunosorbent assay (ELISA), radioimmunoassay (RIA), fluorescence immunoassay (FIA), and fluorescent activated cell sorting (FACS).

### VII. Exemplary Embodiments

Non-limiting embodiments of compositions and methods disclosed herein are as follows:
1. A recombinant Gram-positive cell overexpressing a yvyD gene.
2. A recombinant Gram-positive cell overexpressing a yvyD gene and expressing a gene encoding a protein of interest (POI).
3. The recombinant cell of embodiment 2, expressing multiple copies of a gene encoding a POI.
4. The recombinant cell of any one of embodiments 1-3, wherein the overexpressed yvyD gene comprises at least 50% identity to the yvyD gene of SEQ ID NO: 23.
5. The recombinant cell of any one of embodiments 1-3, wherein the overexpressed yvyD gene comprises at least 50% identity to the yvyD gene coding sequence (CDS) of SEQ ID NO: 18.
6. The recombinant cell of any one of embodiments 1-3, wherein the yvyD gene encodes a protein comprising at least 50% identity to the YvyD protein of SEQ ID NO: 26.
7. The recombinant cell of any one of embodiments 1-3, wherein the yvyD gene encodes a protein comprising at least 50% identity to the RaiA superfamily domain of SEQ ID NO: 27.
8. The recombinant cell of any one of embodiments 1-3, wherein the yvyD gene encodes a protein comprising at least 50% identity to the Ribosome S30AE_C superfamily domain of SEQ ID NO: 28.
9. The recombinant cell of embodiment 2, wherein recombinant cell produces an increased amount of the POI relative to a control cell, when the recombinant and control cells are grown under the same conditions, wherein the control cell expresses the same POI, but does not overexpress the yvyD gene.
10. The recombinant cell of embodiment 9, wherein the control cell expresses its endogenous yvyD gene CDS under the control of its native yvyD gene promoter.
11. The recombinant cell of any one of embodiments 1-3, wherein overexpressing the yvyD gene comprises replacing the native yvyD gene promoter region with a heterologous promoter region operably linked to the downstream (3′) yvyD gene CDS, wherein the heterologous promoter region increases expression of the yvyD gene CDS relative to the native yvyD gene promoter.
12. The recombinant cell of embodiment 11, wherein the heterologous promoter region is selected from a spoVG gene promoter (PspoVG) region comprising at least 90% identity to SEQ ID NO: 21 and a hbs gene promoter (Phbs) region comprising at least 90% identity to SEQ ID NO: 29.
13. The recombinant cell of embodiment 2 or embodiment 3, wherein the POI is an enzyme.
14. The recombinant cell of embodiment 13, wherein the enzyme is selected from the group consisting of acetyl esterases, aminopeptidases, amylases, arabinases, arabinofuranosidases, arylesterases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, lysozymes, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phosphodiesterases, phytases, polyesterases, polygalacturonases, proteases, peptidases, rhamno-galacturonases, ribonucleases, transferases, transglutaminases, xylanases, hexose oxidases, and combinations thereof.
15. The recombinant cell of embodiment 13, wherein the POI is a protease.
16. The recombinant cell of embodiment 15, wherein the protease is a subtilisin.
17. The recombinant cell of embodiment 16, wherein the subtilisin is selected from a native or variant Bacillus lentus subtilisin, a native or variant Bacillus gibsonii subtilisin and a native or variant Bacillus amyloliquefaciens subtilisin.
18. A yvyD expression cassette comprising an upstream (5′) yvyD flanking region (FR) nucleic acid sequence operably linked to a downstream heterologous promoter (het-pro) sequence operably linked to a downstream yvyD gene coding sequence (CDS) operably linked to a downstream (3′) yvyD flanking region (FR) nucleic acid sequence, as generally set forth in Formula 1:

5′-[yvyD FR]-[het-pro]-[yvyD CDS]-[yvyD FR]-3′. Formula 1:
19. A method for producing an increased amount of a protein of interest (POI) in a Gram-positive bacterial cell comprising obtaining a parental cell comprising a yvyD gene having at least 50% identity to the yvyD gene of SEQ ID NO: 23 and genetically modifying the cell to overexpress the yvyD gene.
20. The method of embodiment 19, wherein the parental or modified (recombinant) cell comprise an introduced expression cassette encoding the POI.
21. The method of embodiment 19, wherein the modified cell overexpressing the yvyD gene produces an increased amount of the POI relative to the parental cell when cultivated under the same conditions.
22. The method of embodiment 19, wherein the parental cell yvyD gene comprises at least 50% identity to the yvyD gene coding sequence (CDS) of SEQ ID NO: 18.
23. The method of embodiment 19, wherein the parental cell yvyD gene encodes a YvyD protein comprising at least 50% identity to the YvyD protein of SEQ ID NO: 26.
24. The method of embodiment 23, wherein the YvyD protein comprises at least 50% identity to the RaiA superfamily domain of SEQ ID NO: 27.
25. The method of embodiment 23, wherein the YvyD protein comprises at least 50% identity to the Ribosome S30AE_C superfamily domain of SEQ ID NO: 28.
26. The method of embodiment 19, wherein modified cell overexpressing the yvyD gene comprises a heterologous promoter region operably linked to the downstream (3′) yvyD gene CDS, wherein the heterologous promoter region increases expression of the yvyD gene CDS relative to the native yvyD gene promoter.
27. The method of embodiment 19, wherein modified cell overexpressing the yvyD gene comprises an introduced polynucleotide construct comprising an upstream (5′) heterologous promoter region sequence operably linked to a downstream (3′) yvyD gene CDS comprising at least 50% identity to SEQ ID NO: 18, wherein the heterologous promoter region increases expression of the native yvyD gene CDS relative to the native yvyD gene promoter.
28. The method of embodiment 26 or embodiment 27, wherein the heterologous promoter region is selected from a spoVG gene promoter (PspoVG) region comprising at least 90% identity to SEQ ID NO: 21 and a hbs gene promoter (Phbs) region comprising at least 90% identity to SEQ ID NO: 29.
29. The method of embodiment 19, wherein the POI is an enzyme.
30. The method of embodiment 29, wherein the enzyme is selected from the group consisting of acetyl esterases, aminopeptidases, amylases, arabinases, arabinofuranosidases, arylesterases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, lysozymes, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phosphodiesterases, phytases, polyesterases, polygalacturonases, proteases, peptidases, rhamno-galacturonases, ribonucleases, transferases, transglutaminases, xylanases, hexose oxidases, and combinations thereof.
31. The method of embodiment 29, wherein the POI is a protease.
32. The method of embodiment 31, wherein the protease is a subtilisin.
33. The method of embodiment 19, wherein the Gram-positive bacterial cell is a Bacillus sp. cell.
34. The method of embodiment 33, wherein the Bacillus sp. cell is selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis.
35. The recombinant Gram-positive bacterial cell of embodiment 1 or embodiment 2, wherein the Gram-positive bacterial cell is a Bacillus sp. cell.
36. The recombinant Bacillus sp. cell of embodiment 35, selected from the group consisting of B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis.

### EXAMPLES

Certain aspects of the present invention may be further understood in light of the following examples, which should not be construed as limiting. Modifications to materials and methods will be apparent to those skilled in the art. Standard recombinant DNA and molecular cloning techniques used herein are well known in the art (Ausubel et al., 1987; Sambrook et al., 1989). As described herein, all expression cassettes were transformed into the host strains using the methods described PCT Publication No. WO2019/040412 (incorporated herein by referenced in its entirety).

### Example 1

Construction of yvyD Overexpression Integration Cassettes

The present example describes the construction of yvyD (gene) overexpression (integration) cassettes (e.g., see FIG. 1). More particularly, the yvyD overexpression cassettes described herein were generated by NEBuilder (New England Biolabs) via assembly of PCR amplified DNA fragments. For example, the integration cassette fragments were designed to integrate at the yvzG-yvyD intergene region (hereinafter, “yvzG-yvyD region”) replacing (substituting) the native yvyD promoter with a heterologous promoter, wherein the yvzG-yvyD region flanking sequences were amplified from Bacillus subtilis (e.g., B. subtilis strain 168, ATCC 23857) genomic DNA. As set forth below in TABLE 2, the upstream (5′) yvzG-yvyD flanking region was amplified with oligonucleotide primers 343 (SEQ ID NO: 1) and 402 (SEQ ID NO: 2), and the downstream (3′) yvzG-yvyD flanking region was amplified with oligonucleotide primers 400 (SEQ ID NO: 3) and 370 (SEQ ID NO: 4).

TABLE 2
Oligonucleotide Primers

| SEQ | Primer | Nucleotide Sequence |
|---|---|---|
| 1 | 343 | GAAGGTTATGTAACAGATTTTCG |
| 2 | 402 | TTTTGTGAAAATTCAGTGAACTTTGTC |
| 3 | 400 | TAAAAACGAGCAGGATTTCAGAAAAAATCGTGGAATTGATACACTAATGCTTTTAT |
| | | ATAGGGAAAAGGTGGTGAACTACTATGAACTATAACATCAGAGGAGAAAATATTG |
| 4 | 370 | GGAAACATGCCTGTTACGCGGC |
| 5 | 539 | CATATACATATACCTCCGAACCGCCAATAACAGAGCAAATACAAACAAAATTCGA |
| | | CAAAGTTCACTGAATTTTCACAAAATCGACGGTATCGATAAGCTGGATC |
| 6 | 246 | GGCCTAGGATGCATATGGCGGC |
| 7 | 540 | TAAACCCTTGCATATGTCTAGATAACTTCGTATAATGTATGCTATACGAACGGTAG |
| | | CGGCCGCCATATGCATCCTAGGCCTAAGAAAAGTGATTCTGGGAGAGC |
| 8 | 754 | AGTAGTTCACCACCTTTTCCCTATAT |

A DNA fragment with the spectinomycin antibiotic resistance marker (SpecR) flanked by loxP sites was amplified using oligonucleotide primers 539 (TABLE 2; SEQ ID NO: 5) and 246 (TABLE 2; SEQ ID NO: 6). The spoVG promoter (PspoVG) region was amplified using oligonucleotide primers 540 (TABLE 2; SEQ ID NO: 7) and 754 (TABLE 2; SEQ ID NO: 8). The thirty-six (36) base pairs (bp) of the spoVG promoter region adjacent to the spoVG open reading frame (ORF) that encompassed the Shine-Dalgarno (SD) sequence (see, FIG. 1B) were included adjacent to the promoter regions of the Phbs-yvyD (see, TABLE 3; primer SEQ ID NO: 9).

As shown in TABLE 2, the hbs promoter region was amplified using 675 (SEQ ID NO: 10) and 307 (SEQ ID NO: 11) oligonucleotide primer pairs. The yvyD ORF (SEQ ID NO: 18) was amplified from B. subtilis genomic DNA with oligonucleotide primers 400 (TABLE 2; SEQ ID NO: 3) and 370 (TABLE 2; SEQ ID NO: 4) for PspoVG-yvyD assembly, and primers 674 (TABLE 3; SEQ ID NO: 12) and 370 (TABLE 2, SEQ ID NO: 4) for Phbs-yvyD assembly. NEBuilder assembly was performed as directed by the manufacturer with the overlapping DNA fragments to generate the complete yvzG-yvyD intergene::lox-SpecR-lox-PspoVG-yvyD (FIG. 1A) and yvzG-yvyD intergene::lox-SpecR-lox-Phbs-yvyD (FIG. 1B) integration cassettes. The complete nucleotide sequence of the assembled integration cassette for PspoVG-yvyD is shown in SEQ ID NO: 19, and the complete nucleotide sequence of the assembled integration cassette for Phbs-yvyD is shown in SEQ ID NO: 20.

TABLE 3
Oligonucleotide Primers

| SEQ NO | Primer | Nucleotide Sequence |
|---|---|---|
| 9 | spo VG | TAATGCTTTTATATAGGGAAAAGGTGGTGAACTACT |
| | promoter | |
| | region | |
| 10 | 675 | TAAACCCTTGCATATGTCTAGATAACTTCGTATAATGTATGCTATACGAAC |
| | | GGTAGCGGCCGCCATATGCATCCTAGGCCTAAATCCTTGACGAGCAAGGG |
| | | ATTG |
| 11 | 307 | ATACATTCAGTTCGTTTATTATCATTTC |
| 12 | 674 | GACTGAACTGTGAAGAAATGATAATAAACGAACTGAATGTATCCTAATGC |
| | | TTTTATATAGGGAAAAGGTGGTGAACTACTATGAACTATAACATCAGAGG |
| | | AGAAAATATTG |

### Example 2

Construction and Generation of B. Subtilis Strains that Increase yvyD Expression

The instant example describes the construction of B. subtilis cells (strains) that increase yvyD expression. More particularly, recombinant B. subtilis cells were constructed by the introduction of cassettes which increased expression of the endogenous (native) B. subtilis yvyD gene (SEQ ID NO: 23) by a promoter swap (replacement) integration at the yvzG-yvyD intergene region described in Example 1 and comprising two (2) copies of a gene (2× protease-1; 2× protease-2; 2× protease-3) encoding three (3) different exemplary proteases (2× Protease-1; 2× Protease-2; 2× Protease-3). Isogenic cells which retain the native yvyD promoter and encode the three different exemplary proteases (control cells; 2× Protease-1; 2× Protease-2; 2× Protease-3) were constructed for comparative purposes. For example, about 1-2 μg of the yvzG yvyD intergene::lox-SpecR-lox-PspoVG-yvyD integration cassette (SEQ ID NO: 19) and the yvzG yvyD intergene::lox-SpecR-lox-Phbs-yvyD integration cassette (SEQ ID NO: 20) were separately transformed into a comK competent B. subtilis parental strain.

More particularly, the transformed cells were plated on LB (1% tryptone, 0.5% yeast extract, 1.0% sodium chloride, 1.5% agar) and one-hundred (100) μg/ml spectinomycin, wherein spectinomycin resistant colonies were purified by re-streaking on LB with one-hundred (100) mg/L spectinomycin. The integration of each cassette at the yvzG-yvyD intergene was confirmed by PCR amplification using Q5 High Fidelity PCR polymerase (NEB) and harvested genomic DNA as template with oligonucleotide primers 345 (SEQ ID NO: 12) and 348 (SEQ ID NO: 13) set forth below in TABLE 4, which bind outside of the integration event. Likewise, the correct sequence of each integration cassette was confirmed by Sanger sequencing using oligonucleotides 345 (TABLE 4; SEQ ID NO: 12), 346 (TABLE 4; SEQ ID NO: 14), 300 (TABLE 4; SEQ ID NO: 15), 573 (TABLE 4; SEQ ID NO: 16), 674 (TABLE 3; SEQ ID NO: 11) and 348 (TABLE 4; SEQ ID NO: 13).

TABLE 4
Oligonucleotide Primers

| SEQ NO | Primer | Nucleotide Sequence |
|---|---|---|
| 13 | 345 | GCCATTGAGAATGATGATATGGAAC |
| 14 | 348 | AACCGAGAAACTCGAAAATTTTCCC |
| 15 | 346 | ATCTTCTTTTTACCATCGAAGAGGG |
| 16 | 300 | CCACCTTTTCCCTATATAAAAGCATTA |
| 17 | 573 | GTTTTATGCTTACGGATTTGACGTTC |

Additionally, the spectinomycin antibiotic resistant marker (lox-SpecR-lox) was removed by transformation of a plasmid expressing the Cre recombinase. After plasmid loss, spectinomycin sensitive colonies were identified and the integration cassette was amplified with oligonucleotide primers 346 (TABLE 4; SEQ ID NO: 14) and 573 (TABLE 4, SEQ ID NO: 16). Correct recombination of the lox sites was confirmed for each of the yvyD overexpression strains by sequence analysis using oligonucleotide 346 (TABLE 4; SEQ ID NO: 14). Two (2) cassettes expressing Protease-1 (2× Protease-1) and two (2) cassettes expressing Protease-2 (2× Protease-2) were separately introduced into the PspoVG-yvyD overexpression strain. Likewise, two (2) cassettes expressing Protease-2 (2× Protease-2) and two (2) cassettes expressing Protease-3 (2× Protease-3) were separately introduced into the Phbs-yvyD overexpression strains. Simultaneously, two (2) cassettes expressing Protease-1, Protease-2 and Protease-3, were separately introduced into the parental strain, which expresses yvyD from its native (yvyD) promoter.

### Example 3

yvyD Overexpression Increases Protein Production in B. Subtilis Strains Expressing Two Copies of a Protein of Interest

In the instant example, Applicant assessed the overexpression of yvyD on reporter protease production in the 2 copy protease producing B. subtilis strains described in Example 2 (i.e., 2× Protease-1 and 2× Protease-1 PspoVG-yvyD; 2× Protease-2 and 2× Protease-2 PspoVG-yvyD; 2× Protease-2 and 2× Protease-2 Phbs-yvyD; 2× Protease-3 and 2× Protease-3 Phbs-yvyD). Protease activity assays described herein were performed as set forth in European Patent No. EP0283075 (incorporated herein by reference).

For example, aliquots were taken at time points twelve (12), twenty (20), thirty-six (36), forty-five (45), sixty-one (61), sixty-eight (68), seventy-three (73) and eighty-four (84) hours from the 2× Protease-1 control strain and 2× Protease-1 PspoVG-yvyD strain. A protease activity assay was performed to determine the effect of yvyD increased expression from the spoVG promoter (PspoVG) on the production of Protease-1. The results of the protease assay (FIG. 2) demonstrate that there is a trend towards increased protease production at the end of fermentation, and significant enhancement in protease production at sixty-eight (68) and seventy-three (73) hours, due to yvyD increased expression.

In addition, aliquots were taken at time points sixteen (16), twenty-two (22), thirty-nine (39), forty-six (46), sixty-four (64) and eighty-nine (89) hours from the 2× Protease-2 control strain and 2× Protease-2 PspoVG-yvyD strain. A protease activity assay was performed to determine the effect of yvyD overexpression from the spoVG promoter (PspoVG) on the production of Protease-2. The results of the protease assay (FIG. 3) demonstrate a trend towards increased protease production starting at about thirty-nine (39) hours until the end of fermentation, and significant enhancement in protease production at thirty-nine (39) and forty-six (46) hours, due to yvyD overexpression.

Likewise, aliquots were taken at time points eleven (11), twenty-three (23), thirty-seven (37), fifty (50) and sixty-five (65) hours from the 2× Protease-2 control strain and 2× A Protease-2 Phbs-yvyD strain. A protease activity assay was performed to determine the effect of yvyD overexpression from the hbs promoter (Phbs) on the production of Protease-2. The results of the protease assay (FIG. 4) demonstrate a trend towards increased protease production starting at about thirty-seven (37) hours until the end of fermentation, and a significant enhancement in protease production at thirty-seven (37) hours, due to yvyD overexpression.

Additionally, aliquots were taken at time points fourteen (14), twenty-two (22), thirty-seven (37), forty-six (46), sixty-five (65) and ninety (90) hours from the 2× Protease-3 control strain and 2× Protease-3 Phbs-yvyD strain. A protease activity assay was performed to determine the effect of yvyD overexpression from the hbs promoter (Phbs) on the production of Protease-3. The results of the protease assay (FIG. 5) demonstrate a trend towards increased protease production starting at about twenty-two (22) hours until the end of fermentation, and a significant enhancement in protease production at twenty-two (22) and thirty-seven (37) hours, due to yvyD overexpression.

### REFERENCES

European Patent No. EP0283075
PCT Publication No. WO2014/164777
Beckert et al., “Structure of the Bacillus subtilis hibernating 100S ribosome reveals the basis for 70S dimerization”, The EMBO Journal, 36, pages 2061-2071, 2017.
Drzewiecki et al., “The yvyD Gene of Bacillus subtilis Is Under Dual Control of σ^{B} and σ^{H}”, J. Bacteriology, Vol. 180, No. 24, pages 6674-6680, 1998.
Feaga et al., “Ribosome dimerization protects the small subunit” Journal of Bacteriology, Vol. 202, No. 10, pages 9-20, 2020.
Franklin et al., “Functional Characterization of the Pseudomonas aeruginosa Ribosome Hibernation-Promoting Factor”, Journal of Bacteriology, Volume 202, Issue 19, 2020.
Tagami et al., “Expression of a small (p) ppGpp synthetase, YwaC, in the (p) ppGpp mutant of Bacillus subtilis triggers YvyD-dependent dimerization of ribosome”, Microbiology Open, 1 (2), pages 115-134, 2012.

Zhu and Stülke, “SubtiWiki in 2018: from genes and proteins to functional network annotation of the model organism Bacillus subtilis”, Nucleic Acid Research, Volume 46, Issue D1, Pages D743-D748, 2017.

1. A recombinant Gram-positive cell overexpressing a yvyD gene comprising at least 50% identity to the yvyD gene of SEQ ID NO: 23.

2. The recombinant cell of claim 1 expressing a gene encoding a protein of interest (POI).

3. The recombinant cell of claim 2, expressing multiple copies of the gene encoding the POI.

4. (canceled)

5. The recombinant cell of claim 1, wherein the overexpressed yvyD gene comprises at least 50% identity to the yvyD gene coding sequence (CDS) of SEQ ID NO: 18.

6. The recombinant cell of claim 1, wherein the overexpressed yvyD gene encodes a protein comprising at least 50% identity to the YvyD protein of SEQ ID NO: 26.

7. The recombinant cell of claim 2, wherein recombinant cell produces an increased amount of the POI relative to a control cell expressing the same POI, wherein the control cell does not overexpress the yvyD gene.

8. The recombinant cell of claim 1, wherein overexpressing the yvyD gene comprises replacing the native yvyD gene promoter region with a heterologous promoter region, wherein the heterologous promoter region increases expression of the native yvyD gene CDS relative to the native yvyD gene promoter.

9. The recombinant cell of claim 1, wherein overexpressing the yvyD gene comprises introducing into the cell an expression cassette comprising an upstream heterologous promoter region operably linked to the downstream yvyD gene CDS, wherein the introduced cassette increases expression of the yvyD gene CDS.

10. The recombinant cell of claim 2, wherein the POI is an enzyme.

11. The recombinant cell of claim 1, wherein the Gram-positive bacterial cell is a Bacillus sp. cell.

12. (canceled)

13. A method for producing an increased amount of a protein of interest (POI) in a Gram-positive bacterial cell comprising obtaining a parental cell comprising a yvyD gene having at least 50% identity to the yvyD gene of SEQ ID NO: 23 and genetically modifying the cell to overexpress the yvyD gene.

14. The method of claim 13, wherein the parental cell comprises an introduced expression cassette encoding the POI.

15. The method of claim 13, wherein the modified cell overexpressing the yvyD gene produces an increased amount of the POI relative to the parental cell when cultivated under the same conditions.

16. The method of claim 13, wherein modified cell overexpressing the yvyD gene comprises a heterologous promoter region operably linked to the downstream (3′) yvyD gene CDS, wherein the heterologous promoter region increases expression of the yvyD gene CDS relative to the native yvyD gene promoter.

17. The method of claim 13, wherein the POI is an enzyme.

18. The method of claim 13, wherein the Gram-positive bacterial cell is a Bacillus sp. cell.


SEQUENCE LISTING (29 sequences)

SEQ ID NO: 1 - DNA, 23 bp, synthetic construct
  source 1..23: mol_type = other DNA
gaaggttatgtaacagattttcg

SEQ ID NO: 2 - DNA, 27 bp, synthetic construct
  source 1..27: mol_type = other DNA
ttttgtgaaaattcagtgaactttgtc

SEQ ID NO: 3 - DNA, 111 bp, synthetic construct
  source 1..111: mol_type = other DNA
taaaaacgagcaggatttcagaaaaaatcgtggaattgatacactaatgcttttatatagggaaaaggtggtgaactactatgaactataacatcagaggagaaaatattg

SEQ ID NO: 4 - DNA, 22 bp, synthetic construct
  source 1..22: mol_type = other DNA
ggaaacatgcctgttacgcggc

SEQ ID NO: 5 - DNA, 104 bp, synthetic construct
  source 1..104: mol_type = other DNA
catatacatatacctccgaaccgccaataacagagcaaatacaaacaaaattcgacaaagttcactgaattttcacaaaatcgacggtatcgataagctggatc

SEQ ID NO: 6 - DNA, 22 bp, synthetic construct
  source 1..22: mol_type = other DNA
ggcctaggatgcatatggcggc

SEQ ID NO: 7 - DNA, 104 bp, synthetic construct
  source 1..104: mol_type = other DNA
taaacccttgcatatgtctagataacttcgtataatgtatgctatacgaacggtagcggccgccatatgcatcctaggcctaagaaaagtgattctgggagagc

SEQ ID NO: 8 - DNA, 26 bp, synthetic construct
  source 1..26: mol_type = other DNA
agtagttcaccaccttttccctatat

SEQ ID NO: 9 - DNA, 36 bp, synthetic construct
  source 1..36: mol_type = other DNA
taatgcttttatatagggaaaaggtggtgaactact

SEQ ID NO: 10 - DNA, 105 bp, synthetic construct
  source 1..105: mol_type = other DNA
taaacccttgcatatgtctagataacttcgtataatgtatgctatacgaacggtagcggccgccatatgcatcctaggcctaaatccttgacgagcaagggattg

SEQ ID NO: 11 - DNA, 28 bp, synthetic construct
  source 1..28: mol_type = other DNA
atacattcagttcgtttattatcatttc

SEQ ID NO: 12 - DNA, 111 bp, synthetic construct
  source 1..111: mol_type = other DNA
gactgaactgtgaagaaatgataataaacgaactgaatgtatcctaatgcttttatatagggaaaaggtggtgaactactatgaactataacatcagaggagaaaatattg

SEQ ID NO: 13 - DNA, 25 bp, synthetic construct
  source 1..25: mol_type = other DNA
gccattgagaatgatgatatggaac

SEQ ID NO: 14 - DNA, 25 bp, synthetic construct
  source 1..25: mol_type = other DNA
aaccgagaaactcgaaaattttccc

SEQ ID NO: 15 - DNA, 25 bp, synthetic construct
  source 1..25: mol_type = other DNA
atcttctttttaccatcgaagaggg

SEQ ID NO: 16 - DNA, 27 bp, synthetic construct
  source 1..27: mol_type = other DNA
ccaccttttccctatataaaagcatta

SEQ ID NO: 17 - DNA, 26 bp, synthetic construct
  source 1..26: mol_type = other DNA
gttttatgcttacggatttgacgttc

SEQ ID NO: 18 - DNA, 570 bp, synthetic construct
  source 1..570: mol_type = other DNA
atgaactataacatcagaggagaaaatattgaagtgacacccgcgttaaaggatcatgtcgagaggaagatcggcaagctggagcgctattttgaccatagcgtggatgctgatgtgaacgtcaacttgaagttttacaatgacaaggagtctaaggttgaggttacgattccgatgacagatctggcgcttcggtccgaggtgcataacgaggatatgtacaacgcaattgatctcgcaacaaacaaactggaacgtcaaatccgtaagcataaaacgaaagtaaaccgtaaattccgtgagcagggctctccaaaatatttattggcaaacggtcttggctctgatacagatattgcggttcaggatgacatagaagaggaggagagcttggacatcgtccgtcagaaacgctttaatttaaagccgatggatagtgaagaagcgatcttgcaaatgaatatgctcggccataatttctttgttttcacaaatgcggaaacaaaccttacaaatgtcgtgtaccgcagaaatgacgggaaatatggcttaattgaaccgactgaataa

SEQ ID NO: 19 - DNA, 3209 bp, synthetic construct
  source 1..3209: mol_type = other DNA
gaaggttatgtaacagattttcgcgatgcttggaaacaagccattcaaagtgagcggaaggaccggcacggatcaggcgggatcgcatgaataatatagatcaactatacactgagacgaagagtatgctgtcacacatacaaaatacgccggaaagcgatgaacttttaaagcaaattgaagactttgtggctacacggtctgaactgattcaggagatatctctgccgctttcagaagaagagcgaaagcagatgaaacttattttgacatgggatcagctgattgtgaaggaaatggaaagactgaagcagtcaatcgcaacagaacttcagcagatgaaaagaaaacgtgtgatgcatacgacgtatttaaacccttataacaacattacgattgacggcacatattatgacaagcgtaaataggtgagtgccttgaagagcattgaagattatacgcttttatcagcttattatcatcttctttttaccatcgaagaggggttgtgttatttgatagaggctgaagagaatttatcaaaaacagaaggggatcggatctaccaagatctgatccaagcctttttttatttggattcaacacattcaacacttctcttcattatggattcgcggtgtgctgaaatttgtgtgaaagcatttgaccaaatttttgcggagtttgatcaattggtctctttctcttttccctctcatgagttctgtgagtatttaaaggaacattttctgattcattatagaaaatggatgctgtctattcatcaatgtatggaaccctttttaatcaattaggcgtgtgtgaggtatttgtttcgttcaatcagcatatacatatacctccgaaccgccaataacagagcaaatacaaacaaaattcgacaaagttcactgaattttcacaaaatcgacggtatcgataagctggatcctaccgttcgtataatgtatgctatacgaagttatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatatgtctagataacttcgtataatgtatgctatacgaacggtagcggccgccatatgcatcctaggcctaagaaaagtgattctgggagagccgggatcacttttttatttaccttatgcccgaaatgaaagctttatgacctaattgtgtaactatatcctattttttcaaaaaatattttaaaaacgagcaggatttcagaaaaaatcgtggaattgatacactaatgcttttatatagggaaaaggtggtgaactactatgaactataacatcagaggagaaaatattgaagtgacacccgcgttaaaggatcatgtcgagaggaagatcggcaagctggagcgctattttgaccatagcgtggatgctgatgtgaacgtcaacttgaagttttacaatgacaaggagtctaaggttgaggttacgattccgatgacagatctggcgcttcggtccgaggtgcataacgaggatatgtacaacgcaattgatctcgcaacaaacaaactggaacgtcaaatccgtaagcataaaacgaaagtaaaccgtaaattccgtgagcagggctctccaaaatatttattggcaaacggtcttggctctgatacagatattgcggttcaggatgacatagaagaggaggagagcttggacatcgtccgtcagaaacgctttaatttaaagccgatggatagtgaagaagcgatcttgcaaatgaatatgctcggccataatttctttgttttcacaaatgcggaaacaaaccttacaaatgtcgtgtaccgcagaaatgacgggaaatatggcttaattgaaccgactgaataatgaagagaagccttccgtgatgtccgcggaaggtttttgtttttcttatttgcaaattctttggaaataacaaaaggtatgatatgataatgagaggtatacatggactagtaaattatttatacatgcctctaaaataggcgtgtgatgatagaggagcgttataaatgcttggaattttaaataaaatgtttgatccaacaaaacgtacgctgaatagatacgaaaaaattgctaacgatattgatgcgattcgcggagactatgaaaatctctctgacgacgcattgaaacataaaacaattgaatttaaagagcgtcttgaaaaaggggcgacaacggatgatcttcttgttgaagctttcgctgttgttcgagaagcttcacgccgcgtaacaggcatgtttcc

SEQ ID NO: 20 - DNA, 3286 bp, synthetic construct
  source 1..3286: mol_type = other DNA
gaaggttatgtaacagattttcgcgatgcttggaaacaagccattcaaagtgagcggaaggaccggcacggatcaggcgggatcgcatgaataatatagatcaactatacactgagacgaagagtatgctgtcacacatacaaaatacgccggaaagcgatgaacttttaaagcaaattgaagactttgtggctacacggtctgaactgattcaggagatatctctgccgctttcagaagaagagcgaaagcagatgaaacttattttgacatgggatcagctgattgtgaaggaaatggaaagactgaagcagtcaatcgcaacagaacttcagcagatgaaaagaaaacgtgtgatgcatacgacgtatttaaacccttataacaacattacgattgacggcacatattatgacaagcgtaaataggtgagtgccttgaagagcattgaagattatacgcttttatcagcttattatcatcttctttttaccatcgaagaggggttgtgttatttgatagaggctgaagagaatttatcaaaaacagaaggggatcggatctaccaagatctgatccaagcctttttttatttggattcaacacattcaacacttctcttcattatggattcgcggtgtgctgaaatttgtgtgaaagcatttgaccaaatttttgcggagtttgatcaattggtctctttctcttttccctctcatgagttctgtgagtatttaaaggaacattttctgattcattatagaaaatggatgctgtctattcatcaatgtatggaaccctttttaatcaattaggcgtgtgtgaggtatttgtttcgttcaatcagcatatacatatacctccgaaccgccaataacagagcaaatacaaacaaaattcgacaaagttcactgaattttcacaaaatcgacggtatcgataagctggatcctaccgttcgtataatgtatgctatacgaagttatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatatgtctagataacttcgtataatgtatgctatacgaacggtagcggccgccatatgcatcctaggcctaaatccttgacgagcaagggattgacgctttaaaatgcttgatatggctttttatatgtgttactctacatacagaaattcttcactttgttggacaaacattcctcagagtgcagtttttcttaaaaagccgtttaattgtctttctcttacttgctctcatttttttctgagacaggtttagaatcagactgaactgtgaagaaatgataataaacgaactgaatgtatcctaatgcttttatatagggaaaaggtggtgaactactatgaactataacatcagaggagaaaatattgaagtgacacccgcgttaaaggatcatgtcgagaggaagatcggcaagctggagcgctattttgaccatagcgtggatgctgatgtgaacgtcaacttgaagttttacaatgacaaggagtctaaggttgaggttacgattccgatgacagatctggcgcttcggtccgaggtgcataacgaggatatgtacaacgcaattgatctcgcaacaaacaaactggaacgtcaaatccgtaagcataaaacgaaagtaaaccgtaaattccgtgagcagggctctccaaaatatttattggcaaacggtcttggctctgatacagatattgcggttcaggatgacatagaagaggaggagagcttggacatcgtccgtcagaaacgctttaatttaaagccgatggatagtgaagaagcgatcttgcaaatgaatatgctcggccataatttctttgttttcacaaatgcggaaacaaaccttacaaatgtcgtgtaccgcagaaatgacgggaaatatggcttaattgaaccgactgaataatgaagagaagccttccgtgatgtccgcggaaggtttttgtttttcttatttgcaaattctttggaaataacaaaaggtatgatatgataatgagaggtatacatggactagtaaattatttatacatgcctctaaaataggcgtgtgatgatagaggagcgttataaatgcttggaattttaaataaaatgtttgatccaacaaaacgtacgctgaatagatacgaaaaaattgctaacgatattgatgcgattcgcggagactatgaaaatctctctgacgacgcattgaaacataaaacaattgaatttaaagagcgtcttgaaaaaggggcgacaacggatgatcttcttgttgaagctttcgctgttgttcgagaagcttcacgccgcgtaacaggcatgtttcc

SEQ ID NO: 21 - DNA, 193 bp, synthetic construct
  source 1..193: mol_type = other DNA
taagaaaagtgattctgggagagccgggatcacttttttatttaccttatgcccgaaatgaaagctttatgacctaattgtgtaactatatcctattttttcaaaaaatattttaaaaacgagcaggatttcagaaaaaatcgtggaattgatacactaatgcttttatatagggaaaaggtggtgaactact

SEQ ID NO: 22 - DNA, 234 bp, synthetic construct
  source 1..234: mol_type = other DNA
taaatccttgacgagcaagggattgacgctttaaaatgcttgatatggctttttatatgtgttactctacatacagaaattcttcactttgttggacaaacattcctcagagtgcagtttttcttaaaaagccgtttaattgtctttctcttacttgctctcatttttttctgagacaggtttagaatcagactgaactgtgaagaaatgataataaacgaactgaatgtatcc

SEQ ID NO: 23 - DNA, 648 bp, Bacillus subtilis
  source 1..648: mol_type = other DNA
gatttatgtttcagcaggaattgtaaagggtaaaagagaaatagatacatatccttaataaaagggaggcgttctttgatgaactataacatcagaggagaaaatattgaagtgacacccgcgttaaaggatcatgtcgagaggaagatcggcaagctggagcgctattttgaccatagcgtggatgctgatgtgaacgtcaacttgaagttttacaatgacaaggagtctaaggttgaggttacgattccgatgacagatctggcgcttcggtccgaggtgcataacgaggatatgtacaacgcaattgatctcgcaacaaacaaactggaacgtcaaatccgtaagcataaaacgaaagtaaaccgtaaattccgtgagcagggctctccaaaatatttattggcaaacggtcttggctctgatacagatattgcggttcaggatgacatagaagaggaggagagcttggacatcgtccgtcagaaacgctttaatttaaagccgatggatagtgaagaagcgatcttgcaaatgaatatgctcggccataatttctttgttttcacaaatgcggaaacaaaccttacaaatgtcgtgtaccgcagaaatgacgggaaatatggcttaattgaaccgactgaataa

SEQ ID NO: 24 - DNA, 78 bp, synthetic construct
  source 1..78: mol_type = other DNA
gatttatgtttcagcaggaattgtaaagggtaaaagagaaatagatacatatccttaataaaagggaggcgttctttg

SEQ ID NO: 25 - DNA, 36 bp, synthetic construct; identical to SEQ ID NO: 9
  source 1..36: mol_type = other DNA

SEQ ID NO: 26 - protein, 189 aa, synthetic construct
MNYNIRGENIEVTPALKDHVERKIGKLERYFDHSVDADVNVNLKFYNDKESKVEVTIPMTDLALRSEVHNEDMYNAIDLATNKLERQIRKHKTKVNRKFREQGSPKYLLANGLGSDTDIAVQDDIEEEESLDIVRQKRFNLKPMDSEEAILQMNMLGHNFFVFTNAETNLTNVVYRRNDGKYGLIEPTE

SEQ ID NO: 27 - protein, 111 aa, synthetic construct
MNYNIRGENIEVTPALKDHVERKIGKLERYFDHSVDADVNVNLKFYNDKESKVEVTIPMTDLALRSEVHNEDMYNAIDLATNKLERQIRKHKTKVNRKFREQGSPKYLLAN

SEQ ID NO: 28 - protein, 52 aa, synthetic construct
IVRQKRFNLKPMDSEEAILQMNMLGHNFFVFTNAETNLTNVVYRRNDGKYGL

SEQ ID NO: 29 - DNA, 270 bp, synthetic construct
  source 1..270: mol_type = other DNA
taaatccttgacgagcaagggattgacgctttaaaatgcttgatatggctttttatatgtgttactctacatacagaaattcttcactttgttggacaaacattcctcagagtgcagtttttcttaaaaagccgtttaattgtctttctcttacttgctctcatttttttctgagacaggtttagaatcagactgaactgtgaagaaatgataataaacgaactgaatgtatcctaatgcttttatatagggaaaaggtggtgaactact